(19) United States
(12) Patent Application Publication
(10) Pub. No.: US 2006/0009527 A1
(43) Pub. Date: Jan. 12, 2006

(54) Method of treating TRX mediated diseases

(76) Inventors: Victoria M. Richon, Rye, NY (US); Paul A. Marks, Washington, CT (US); Richard A. Rifkind, New York, NY (US); Lisa M. Butler, Athelstone (AU)

Correspondence Address: MINTZ LEVIN, COHEN, FERRIS, 24th Floor, 666 Third Avenue, New York, NY, 10017, US

(21) Appl. No.: 11/223,547

(22) Filed: Sep. 9, 2005

Related U.S. Application Data
(63) Continuation of application No. 10/369,094, filed on Feb. 14, 2003, now abandoned.
(60) Provisional application No. 60/357,383, filed on Feb. 15, 2002.

(51) Int. Cl. A61K 31/19

(52) U.S. Cl. 514/575

(57) ABSTRACT

The present invention provides a novel method for treating and/or preventing thioredoxin (TRX)-mediated diseases and conditions, by administering to a subject in need of such treatment a therapeutically effective amount of a histone deacetylase (HDAC) inhibitor or a pharmaceutically acceptable salt or hydrate thereof. The HDAC inhibitor can alter the expression of a thioredoxin-binding-protein (e.g. TBP-2), which in turn can lead to an altered TRX/thioredoxin-binding-protein cellular binding interaction, resulting in an increase or decrease in the level or activity of cellular TRX, for example the expression level or reducing activity of TRX. Thus the present invention relates to the use of HDAC inhibitors in a method of preventing and/or treating a wide variety of thioredoxin (TRX)-mediated diseases and conditions, such as inflammatory diseases, allergic diseases, autoimmune diseases, diseases associated with oxidative stress or diseases characterized by cellular hyperproliferation.

### RELATED APPLICATION

This application claims the benefit of U.S. Provisional Application No. 60/357,383, filed on Feb. 15, 2002. The entire teachings of the above application are incorporated herein by reference.

### GOVERNMENT SUPPORT

The invention was supported, in whole or in part, by NIH grants CA-0974823, U01 CA-84292 and NCI Core Grant No. 08748. The Government has certain rights in the invention.

### BACKGROUND OF THE INVENTION

Thioredoxin (TRX) is a 12 kDa, ubiquitous multifunctional protein with the conserved active site sequence: -Cys-Gly-Pro-Cys- that forms a disulfide in the oxidized form or a dithiol in the reduced form. TRX plays an important biological role both in intra- and extracellular compartments. Nakamura et al. have reported that TRX is an intracellular redox protein with extracellular cytokine-like and chemokine-like activities (Nakamura, H. et al., PNAS, 98(5):2688-2693, 2001). This general protein dithiol-disulfide oxidoreductase, can operate in a wide variety of intracellular processes either independently or together with NADPH and thioredoxin reductase (TR) as part of the TRX-TR system. In its reduced form, TRX is a hydrogen donor for ribonucleotide reductase essential for DNA synthesis and a general protein disulfide reductase involved in redox regulation. TRX plays an important role in the maintenance of an appropriate intracellular reduction/oxidation (redox) balance which is of crucial importance for normal cellular functioning that involves cell viability, signaling, activation, and proliferation. For example, TRX has been shown to be involved in the redox regulation of the transcription factors such as, NF-κB and AP-1.

TRX plays a key biological role in cellular redox reactions, and accordingly abnormal levels of this protein have been found in numerous pathophysiological and disease states. For example, the expression of TRX can be enhanced by various types of stress and as such TRX is a stress-inducible protein. There has been accumulating evidence that TRX is induced and released from cells by a variety of oxidative stress conditions (Nakashima et al., Liver 2001, 21, 295-299 and references cited therein). TRX can behave as a scavenger of reactive oxygen intermediates (ROI), and as such, can offer protection against cytotoxicity, in which the generation of ROI can play a part in the cytotoxic mechanism. Recently it was reported that TRX induction in rats is accompanied with ROI overproduction and that TRX can play an important role not only in scavenging ROI but also in signal transduction during ischemia (Takagi et al. Neuroscience Letters (1998), 251, 25-28).

Moreover, an increase in oxidative stress is thought to be involved in the progression of heart disease. It has recently been shown that serum levels of TRX in patients with heart failure is significantly higher than in control subject, indicating a possible association between TRX levels and the severity of heart failure (Kisiroto et al., Jpn. Cir. J. (2001), 65(6), 491-494).

Elevated levels of TRX have also been linked with chronic and/or malignant liver diseases. Miyazaki et al. reported that serum level of TRX is increased significantly in patients with hepatocellular carcinoma (Miyazaki et al., Oxid. Stress Dis. (1999), 3, 235-250). Furthermore, serum TRX levels have been found to be indicative of oxidative stress in patients with hepatitis C virus infection (J. Hepatol. (2000) 33: 616-622).

Elevated levels of TRX have also been found in cancer. That is, TRX can stimulate proliferation of a wide variety of cancer cell lines and inhibit apoptosis in cells overexpressing the protein.

In addition, TRX has recently been shown to be a potent chemotactic protein with potency comparable to other known chemokines, indicating a pathogenic role of TRX in infection and inflammation (Bertini, R. et al., J. of Exp. Med., 189(11):1783-1789, 1999). Since TRX production is induced by oxidants, a link between oxidative stress and inflammation is established. Indeed, TRX has been implicated in various inflammatory and autoimmune diseases. For example, it has been reported that the concentration of TRX in the synovial fluid and synovial tissue of patients suffering from rheumatoid arthritis (RA) is significantly increased and that based on the growth-promoting and cytokine-like properties the increased expression of TRX can contribute to the disease activity in RA (Maurice, M. et al., Arthritis & Rheumatism, 42(11):2430-2439, 1999). Furthermore, increased TRX levels have been reported in HIV disease (Nakamura et al., Int. Immunol. 8: 603-611, 1996).

Recently, a TRX-binding protein designated as thioredoxin-binding protein-2 (TBP-2), was identified (Nishiyama, A. et al., J. Biol. Chem., 274(31):21645-50, 1999). The TBP-2 is identical to vitamin D(3) up-regulated protein 1 (VDUP1). The association of TRX with TBP-2NVDUP1 was observed both in vitro and in vivo, showing that the TRX-TBP-2NVDUP1 interaction can affect the redox regulatory mechanism in cellular processes. In addition, it was shown that TBP-2/VDUP1 bound to reduced TRX but not to oxidized TRX. Importantly, it has been shown that both reducing activity and expression of TRX is inhibited by association with TBP-2. Thus an induction in the expression of TBP-2 is associated with inhibition of both the biological function and expression of TRX.

The ability of TRX to induce inflammation, inhibit apoptosis, and act as a growth factor, and the involvement of TRX in various disease states such as inflammatory and autoimmune diseases and conditions involving oxidative stress, make it an attractive target for the treatment of disorders characterized by an altered level of TRX. Thus, there is a need in the art to identify compounds that are effective at modulating TRX.

### SUMMARY OF THE INVENTION

The present invention provides a novel method for treating and/or preventing thioredoxin (TRX)-mediated diseases and conditions, by administering to a subject in need of such treatment a therapeutically effective amount of a histone deacetylase (HDAC) inhibitor or a pharmaceutically acceptable salt or hydrate thereof. The HDAC inhibitor can alter the expression of a thioredoxin-binding-protein (e.g. thioredoxin-binding-protein-2 or TBP-2), which in turn can lead to an altered TRX/thioredoxin-binding-protein cellular binding interaction, resulting in an increase or decrease in the level (e.g. expression level) or activity (e.g. reducing activity) of cellular TRX. Thus the present invention relates to the use of HDAC inhibitors in a method of preventing and/or treating a wide variety of thioredoxin (TRX)-mediated diseases and conditions, such as inflammatory diseases, allergic diseases, autoimmune diseases, diseases associated with oxidative stress or diseases characterized by cellular hyperproliferation.

The present invention is based upon the unexpected discovery that compounds capable of inhibiting histone deacetylases (HDACs) can induce expression of a thioredoxin-binding-protein such as thioredoxin-binding-protein-2 (TBP-2). This induction of the thioredoxin-binding-protein is associated with a decrease in the level or activity of thioredoxin (TRX) resulting from interaction of TRX with the thioredoxin-binding-protein.

As such, compounds capable of inhibiting histone deacetylases (HDAC inhibitors) can be used in treating TRX-mediated diseases and conditions, for example TRX-mediated diseases which are characterized by an altered level or activity of TRX. For example, the HDAC inhibitors can be effective at treating the TRX-mediated diseases by modulating the level or activity of TRX, e.g., causing a decrease or increase in the level or activity of TRX. For example, when the TRX-mediated disease is characterized by an increased level or activity of TRX, the HDAC inhibitor can decrease the level or activity of TRX.

By “level” is meant any one or more of the following: expression level, gene expression level (m-RNA), protein expression level, or any combination thereof, which can be observed in vitro or in vivo.

By “activity” is meant any one or more of the following: reducing activity, i.e. the ability of TRX to participate in cellular redox reactions, enzymatic activity or any combination thereof, which can be observed in vitro or in vivo.

Thus, in one embodiment, the present invention provides a method for treating and/or preventing thioredoxin (TRX)-mediated diseases and conditions, by administering to a subject in need of such treatment a therapeutically effective amount of a histone deacetylase (HDAC) inhibitor or a pharmaceutically acceptable salt or hydrate thereof.

Non-limiting examples of TRX-mediated diseases are inflammatory diseases, allergic diseases, autoimmune diseases, disease associated with oxidative stress or diseases characterized by cellular hyperproliferation. Specific examples of such diseases include but are not limited to: inflammatory conditions of the joint; rheumatoid arthritis (RA); psoriatic arthritis; inflammatory bowel diseases such as Crohn's disease and ulcerative colitis; spondyloarthropathies; scleroderma; psoriasis; inflammatory dermatoses such an dermatitis, eczema, atopic dermatitis and allergic contact. dermatitis; urticaria; vasculitis; eosinphilic myositis; eosinophilic fasciitis; cancers with leukocyte infiltration of the skin or organs; ischemic injury; cerebral ischemia; HIV; heart failure; chronic, acute or malignant liver disease; autoimmune thyroiditis; systemic lupus erythematosus; Sjorgren's syndrome; lung diseases; acute pancreatitis; amyotrophic lateral sclerosis (ALS); Alzheimer's disease; cachexia/anorexia; asthma; atherosclerosis; chronic fatigue syndrome; fever; diabetes; glomerulonephritis; graft versus host rejection; hemohorragic shock; hyperalgesia; multiple sclerosis; myopathies; osteoporosis; Parkinson's disease; pain; pre-term labor; psoriasis; reperfusion injury; cytokine-induced toxicity; side effects from radiation therapy; temporal mandibular joint disease; tumor metastasis; an inflammatory condition resulting from strain, sprain, cartilage damage, trauma such as burn, orthopedic surgery, infection or other disease processes; respiratory allergic diseases such as asthma, allergic rhinitis, hypersensitivity lung diseases, hypersensitivity pneumonitis, eosinophilic pneumonias, delayed-type hypersensitivity and interstitial lung diseases (ILD); systemic anaphylaxis or hypersensitivity responses; drug allergies and insect sting allergies.

In another embodiment, the present invention provides a method of modulating the level or activity of thioredoxin (TRX) in a subject, comprising the step of administering to the subject a histone deacetylase (HDAC) inhibitor, or a pharmaceutically acceptable salt or hydrate thereof, in an amount effective to modulate the level or activity of TRX in the subject. The terms “level” and “activity” have one or more of the definitions recited above.

In another embodiment, the present invention provides a method of modulating the level or activity of thioredoxin (TRX) in a cell, comprising the step of contacting the cell with a histone deacetylase (HDAC) inhibitor, or a salt or hydrate thereof, in an amount effective to modulate the level or activity of TRX in the cell. The terms “level” and “activity” have one or more of the definitions recited above.

In yet another embodiment, the present invention provides a method of modulating the level of a thioredoxin-binding protein in a cell, comprising the step of contacting the cell with a histone deacetylase (HDAC) inhibitor, or a salt or hydrate thereof, in an amount effective to modulate the level of the thioredoxin-binding-protein in the cell. “Level” has any one or more of the definitions recited above.

In one particular embodiment, the HDAC inhibitor increases the level of the thioredoxin-binding-protein by inducing expression of the thioredoxin-binding-protein gene or protein. This induction of the thioredoxin-binding-protein can result in a decrease in the level or activity of TRX resulting from increased TRX/thioredoxin-binding-protein binding interaction. In one particular embodiment, the thioredoxin-binding-protein is TBP-2 (thioredoxin-binding-protein-2). “Level ” and “activity” have any one or more of the definitions recited above.

HDAC inhibitors which are effective at treating and/or preventing TRX-mediated diseases, and which can be used in the methods of the present invention, include but are not limited to hydroxamic acid derivatives, Short Chain Fatty Acids (SCFAs), cyclic tetrapeptides, benzamide derivatives, or electrophilic ketone derivatives, as defined herein.

Specific non-limiting examples of HDAC inhibitors suitable for use in the methods of the present invention are:
A) Hydroxamic acid derivatives selected from SAHA, pyroxamide, CBHA, Trichostatin A (TSA), Trichostatin C, Salicylihydroxamic Acid (SBHA), Azelaic Bishydroxamic Acid (ABHA), Azelaic-1-Hydroxamate-9-Anilide (AAHA), 6-(3-Chlorophenylureido) carpoic Hydroxamic Acid (3C1-UCHA), Oxamflatin, A-161906, Scriptaid, PXD-101, LAQ-824, CHAP, MW2796, and MW2996;
B) Cyclic tetrapeptides selected from, Trapoxin A, FR901228 (FK 228, Depsipeptide), FR225497, Apicidin, CHAP, HC-Toxin, WVF27082, and Chlamydocin;
C) Short Chain Fatty Acids (SCFAs) selected from Sodium Butyrate, Isovalerate, Valerate, 4 Phenylbutyrate (4-PBA), Phenylbutyrate (PB), Propionate, Butyramide, Isobutyramide, Phenylacetate, 3-Bromopropionate, Tributyrin, Valproic acid and Valproate;
D) Benzamide derivatives selected from CI-994, MS-27-275 (MS-275) and a 3′-amino derivative of MS-27-275;
E) Electrophilic ketones derivative selected from a trifluoromethyl ketone and an a-ketoamide such as an N-methyl-a-ketoamide; and
F) Depudecin.

Preferred HDAC inhibitors include:
Suberoylanilide hydroxamic acid (SAHA) or a pharmaceutically acceptable salt or hydrate thereof which is represented by the following structural formula:

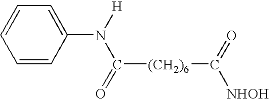

Pyroxamide or a pharmaceutically acceptable salt or hydrate thereof which is represented by the following structural formula: 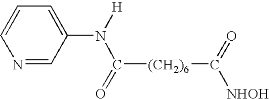
m-carboxycinnamic acid bishydroxamate (CBHA) or a pharmaceutically acceptable salt or hydrate thereof which is represented by the structural formula:

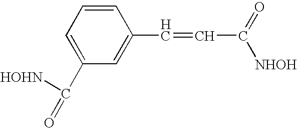

Other non-limiting examples of HDAC inhibitors which are suitable for use in the methods of the present invention are:

A compound represented by the structure: 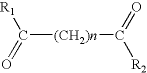
wherein R₁ and R₂ can be the same or different; when R₁ and R₂ are the same, each is a substituted or unsubstituted arylamino, cycloalkylamino, pyridineamino, piperidino, 9-purine-6-amine or thiazoleamino group; when R₁ and R₂ are different R₁═R₃—N—R₄, wherein each of R₃ and R₄ are independently the same as or different from each other and are a hydrogen atom, a hydroxyl group, a substituted or unsubstituted, branched or unbranched alkyl, alkenyl, cycloalkyl, aryl alkyloxy, aryloxy, arylalkyloxy or pyridine group, or R₃ and R₄ are bonded together to form a piperidine group, R₂ is a hydroxylamino, hydroxyl, amino, alkylamino, dialkylamino or alkyloxy group and n is an integer from about 4 to about 8 or a pharmaceutically acceptable salt or hydrate thereof.

A compound represented by the structure: 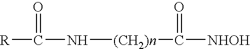
wherein R is a substituted or unsubstituted phenyl, piperidine, thiazole, 2-pyridine, 3-pyridine or 4-pyridine and n is an integer from about 4 to about 8 or a pharmaceutically acceptable salt or hydrate thereof.

A compound represented by the structure: 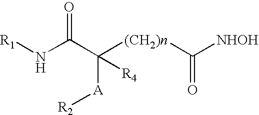
wherein A is an amide moiety, R₁ and R₂ are each selected from substituted or unsubstituted aryl, naphtha, pyridineamino, 9-purine-6-amine, thiazoleamino, aryloxy, arylalkyloxy or pyridine, R₄ is hydrogen, a halogen, a phenyl or a cycloalkyl moiety and n is an integer from 3 to 10 or a pharmaceutically acceptable salt or hydrate thereof.

The present invention thus provides a safe and effective method of preventing and/or treating a wide variety of thioredoxin (TRX)-mediated diseases and conditions, especially diseases characterized by an altered cellular level or activity of TRX, such as inflammatory diseases, allergic diseases, autoimmune diseases, diseases associated with oxidative stress or disease characterized by cellular hyperproliferation. The methods comprise administering a therapeutically effective amount of one or more of a wide selection of HDAC inhibitors as described herein.

### BRIEF DESCRIPTION OF THE DRAWINGS

The foregoing and other objects, features and advantages of the invention will be apparent from the following more particular description of preferred embodiments of the invention, as illustrated in the accompanying drawings.

FIG. 1 is a picture of a Northern blot of TBP-2 mRNA from LNCaP human prostate cells and T24 bladder carcinoma cells cultured with SAHA at the indicated concentrations or vehicle alone (control) for 0.5, 2, 4, 6, 12 and 24 hours. A 1.1 kb, ³²P-labelled TBP-2 cDNA probe was used (upper panel for each cell line). Blots were re-hybridized with a g-32P-labelled 18S oligonucleotide probe to indicate RNA loading and are shown in the lower panel for each cell line. The results show that TBP-2 mRNA in transformed cells is induced by SAHA.

FIG. 2A is picture of a multiple tissue Northern blot showing poly A+ RNA from the indicated normal tissues (Clontech) which were hybridized with a 1.1 kb ³²P-labelled TBP-2 cDNA probe (upper panel). The blots were re-hybridized with a 2.0 kb probe for β-actin, as a control for loading (lower panel). The results show that TBP-2 is expressed in normal tissues.

FIG. 2B is picture of a dot blot containing matched samples of cDNA samples extracted normal human tissues and tumors (Clontech) which were hybridized with a 1.1 kb ³²P-labelled TBP-2 cDNA probe. Samples of colon and breast tumors (T) are shown, with the cDNA from the normal tissue (N) shown directly above each corresponding tumor sample. The results show that TBP-2 is expressed at lower levels in tumor tissue compared to normal tissue.

FIG. 3 is a picture of a Northern blot showing the expression of TBP-2 mRNA and thioredoxin mRNA in T24 human bladder carcinoma cells cultured with SAHA at 2.5 μM and 5.0 μM and with vehicle alone (0) for the indicated time (hrs). A 500 bp ³²P-labelled cDNA probe was used to detect TRX (upper panel). The blots were subsequently re-hydribidized with the 1.1 kb 32P-labelled TBP-2 cDNA probe to confirm induction of TBP-2 (middle panel) and a γ-³²P-labelled 18S oligonucleotide probe to indicate RNA loading (lower panel). The results show that the expression of thioredoxin is reduced in transformed cells cultured with SAHA.

FIG. 4 is the nucleotide sequence of the 5′ untranslated region and promoter of the TBP-2 gene. The adenine in the translation initiation codon, which is indicated in bold and underlined type, has been designated “+1”. The TATA box is indicated in bold, underlined type. The putative binding sites for transcription factors are shown in bold italicized type. The 1763 bp “full-length” region of the promoter used for the reporter gene assays contains nucleotides −264 to −2026 (relative to the translation initiation codon in this sequence.

FIG. 5A is a graph showing the luminescence of 293T cells which were transfected with 100 ng of an empty PGL2 vector, a pGL2-SV40 positive control vector or the TBP-2 construct (−2026), 24 hours after transfection. The results show that the TBP-2 promoter is functional.

FIG. 5B is a graph showing the fold induction of 293T cells which were transfected with 100 ng of an empty PGL2 vector, a pGL2-SV40 positive control vector or the TBP-2 construct (−2026) and incubated with medium containing DMSO or SAHA (0.5, 1 or 2 μM) 12 hours after transfection. Luminescence was measured at 24 hours after transfection and normalized for total protein concentration of each sample. Fold induction is obtained by normalizing the luciferase value in the presence of SAHA against the luciferase value in the absence of SAHA (FIG. 5A). The results show that TBP-2 promoter activity is induced by SAHA.

FIG. 6A is a schematic representation of the putative TBP-2 promoter region and the deletion mutants. The positions of putative transcription factors binding sites in the promoter are shown, 1: NF-kB binding site, 2: vitamin D receptor/retinoid X receptor responsive element, 3: E2F binding site, 4: E Box, 5: inverted CCAAT box, 6: CCAAT box, 7: E box and 8: TATA box.

FIG. 6B is a graph of the luciferase activity of 293T cells which were transfected with constructs prepared from different lengths of the 5′-flanking region of human TBP-2 gene amplified by PCR and cloned upstream of the luciferase gene in the PGL-2 vector. The results shown (+/−standard deviation) are the mean of three independent transfections normalized against total protein.

FIG. 6C is a graph showing fold induction of 293T cells which were transfected as described in 6B and incubated with 2 μM SAHA twelve hours after transfection. Luciferase activity was normalized against total protein and fold induction was calculated as described for FIG. 5B above. The results show that SAHA induces TBP-2 promoter activity.

FIG. 6D is a graph showing fold induction of 293T cells which were transfected with a construct prepared from a mutant TBP-2 promoter (mutated at the inverted CCAAT box, see FIG. 4) cloned into PGL-2 and transfected for 12 hours. After 12 hours the cells were cultured with SAHA (2 μM) for 12 hours or were maintained without treatment for 12 hours. Fold induction was calculated as described for FIG. 5B above. The results show that the inverted CCAAT box is necessary for SAHA inducibility.

FIG. 7A is a picture of an electrophoretic mobility-shift get demonstrating the role of NF-Y in induction of TBP-2. Binding of NF-Y to the inverted CCAAT box in TBP-2 promoter. Electrophoretic mobility-shift assay (lanes 1-4 and 8-10) detects specific complex formation at the inverted CCAAT box. 32P-labeled wild-type probe (20,000 cpm, ⁻0.5 ng; lane 1) was incubated with 10 mg nuclear extracts prepared from untreated (lanes 2-7) or 7.5 μM SAHA-treated (12 h) (lanes 8-13) T24 cells, in the absence (lanes 2 and 8) or presence of 25 ng (×50) wild-type (lanes 3 and 9) or mutant (lanes 4 and 10) oligonucleotide competitors. For supershift assays, nuclear extracts were incubated with 2 mg rabbit anti-NF-YA (lanes 5 and 11), 2 mg goat anti-C/EBP (lanes 6 and 12), or 2 μg normal rabbit IgG (lanes 7 and 13). WT, wild type probe competitor; Mut, mutant probe competitor; YA, anti-NF-YA; C/E, anti-C/EBP.

FIG. 7B is a graph showing that the dominant negative NF-Y mutant (NF-YA29) decreases the promoter induction by SAHA. The pGL2-TBP-2-2026 promoter construct (100 ng) was cotransfected with NF-YA29 expression vector as indicated, and then treated with or without SAHA (2 μM) for 24 hr.

### DETAILED DESCRIPTION OF THE INVENTION

A description of preferred embodiments of the invention follows. The present invention provides a novel method for treating and/or preventing thioredoxin (TRX)-mediated diseases and conditions, by administering to a subject in need of such treatment a therapeutically effective amount of a histone deacetylase (HDAC) inhibitor or a pharmaceutically acceptable salt or hydrate thereof. The HDAC inhibitor can alter the expression of a thioredoxin-binding-protein (e.g. thioredoxin-binding-protein-2 or TBP-2), which in turn can lead to an altered TRX/thioredoxin-binding-protein cellular binding interaction, resulting in an increase or decrease in the level (e.g. expression level) or activity (e.g. redox activity) of cellular TRX. Thus the present invention relates to the use of HDAC inhibitors in a method of preventing and/or treating a wide variety of thioredoxin (TRX)-mediated diseases and conditions, such as inflammatory diseases, allergic diseases, autoimmune diseases, diseases associated with oxidative stress or diseases characterized by cellular hyperproliferation.

The present invention is based upon the unexpected discovery that compounds capable of inhibiting histone deacetylases (HDACs) can alter expression of a thioredoxin-binding-protein, i.e. increase or decrease expression of the thioredoxin-binding-protein. As demonstrated herein, it has been unexpectedly and surprisingly discovered that compounds capable of inhibiting histone deacetylases can induce expression of the TBP-2 gene. This induction of the TBP-2 gene can result in a decrease in the level of TRX resulting from interaction of the TRX with TBP-2. Specifically, it has been determined, employing microarray analysis, that the histone deacetylase inhibitor SAHA can induce the expression of the thioredoxin-binding protein-2 (TBP-2) gene in LNCaP prostate cells, and MCF-7 and MDA-MB-468 breast cells. The induction of TBP-2 was associated with a decrease in thioredoxin (TRX) mRNA levels in these cells.

As such, compounds capable of inhibiting histone deacetylases can be used in treating TRX-mediated diseases and conditions, for example TRX-mediated disease which are characterized by an altered level or activity of TRX. Without wishing to be bound to any particular theory, one mechanism by which the HDAC inhibitor is effective at treating the TRX-mediated diseases is by modulating the level or activity of TRX, i.e. causing a decrease or increase in the level or activity of TRX. For example, when the TRX-mediated disease is characterized by an increased level or activity of TRX, the HDAC inhibitor decreases the level or activity of TRX.

By “level” is meant any one or more of the following: expression level, gene expression level (m-RNA), protein expression level, or any combination thereof, which can be observed in vitro or in vivo.

By “activity” is meant any one or more of the following: reducing activity, i.e. the ability of TRX to participate in cellular redox reactions, enzymatic activity or any combination thereof, which can be observed in vitro or in vivo.

Thus, in one embodiment, the present invention provides a novel method for treating and/or preventing thioredoxin (TRX)-mediated diseases and conditions, by administering to a subject in need of such treatment a therapeutically effective amount of a histone deacetylase (HDAC) inhibitor or a pharmaceutically acceptable salt or hydrate thereof.

In another embodiment, the present invention provides a method of modulating the level or activity of thioredoxin (TRX) in a subject, comprising the step of administering to the subject a histone deacetylase (HDAC) inhibitor, or a pharmaceutically acceptable salt or hydrate thereof, in an amount effective to modulate the level or activity of TRX in the subject. The terms “level” and “activity” have one or more of the definitions recited above.

In another embodiment, the present invention provides a method of modulating the level or activity of thioredoxin (TRX) in a cell, comprising the step of contacting the cell with a histone deacetylase (HDAC) inhibitor, or a salt or hydrate thereof, in an amount effective to modulate the level or of TRX in the cell. The terms “level” and “activity” have one or more of the definitions recited above.

In yet another embodiment, the present invention provides a method of modulating the level of a thioredoxin-binding protein in a cell, comprising the step of contacting the cell with a histone deacetylase (HDAC) inhibitor, or a salt or hydrate thereof, in an amount effective to modulate the level of the thioredoxin-binding-protein in the cell. “Level” has any one or more of the definitions recited above.

In one particular embodiment, the HDAC inhibitor increases the level of the thioredoxin-binding-protein by inducing expression of the thioredoxin-binding-protein gene or protein. This induction of thioredoxin-binding-protein can result in a decrease in the level or activity of TRX resulting from increased TRX/thioredoxin-binding-protein binding interaction. In one particular embodiment, the thioredoxin-binding-protein is TBP-2 (thioredoxin-binding-protein-2). “Level” and “activity” have any one or more of the definitions recited above.

Histone Deacetylases and Histone Deacetylase Inhibitors

Histone deacetylases (HDACs) as that term is used herein are enzymes which catalyze the removal of acetyl groups from lysine residues in the amino terminal tails of the nucleosomal core histones. As such, HDACs together with histone acetyl transferases (HATs) regulate the acetylation status of histones. Histone acetylation affects gene expression and inhibitors of HDACs, such as the hydroxamic acid-based hybrid polar compound suberoylanilide hydroxamic acid (SAHA) induce growth arrest, differentiation and/or apoptosis of transformed cells in vitro and inhibit tumor growth in vivo. HDACs can be divided into three classes based on structural homology. Class I HDACs (HDACs 1, 2, 3 and 8) bear similarity to the yeast RPD3 protein, are located in the nucleus and are found in complexes associated with transcriptional co-repressors. Class II HDACs (HDACs 4, 5, 6, 7 and 9) are similar to the yeast HDA1 protein, and have both nuclear and cytoplasmic subcellular localization. Both Class I and II HDACs are inhibited by hydroxamic acid-based HDAC inhibitors, such as SAHA. Class III HDACs form a structurally distant class of NAD dependent enzymes that are related to the yeast SIR2 proteins and are not inhibited by hydroxamic acid-based HDAC inhibitors.

Histone deacetylase inhibitors or HDAC inhibitors, as that term is used herein are compounds which are capable of inhibiting the deacetylation of histones in vivo, in vitro or both. As such, HDAC inhibitors inhibit the activity of at least one histone deacetylase. As a result of inhibiting the deacetylation of at least one histone, an increase in acetylated histone occurs and accumulation of acetylated histone is a suitable biological marker for assessing the activity of HDAC inhibitors. Therefore, procedures which can assay for the accumulation of acetylated histones can be used to determine the HDAC inhibitory activity of compounds of interest. It is understood that compounds which can inhibit histone deacetylase activity can also bind to other substrates and as such can inhibit other biologically active molecules such as enzymes.

For example, in patients receiving HDAC inhibitors, the accumulation of acetylated histones in peripheral mononuclear cells as well as in tissue treated with HDAC inhibitors can be determined against a suitable control.

HDAC inhibitory activity of a particular compound can be determined in vitro using, for example, an enzymatic assays which shows inhibition of at least one histone deacetylase. Further, determination of the accumulation of acetylated histones in cells treated with a particular composition can be determinative of the HDAC inhibitory activity of a compound.

Assays for the accumulation of acetylated histones are well known in the literature. See, for example, Marks, P. A. et al., J. Natl. Cancer Inst., 92:1210-1215, 2000, Butler, L. M. et al., Cancer Res. 60:5165-5170 (2000), Richon, V. M. et al., Proc. Natl. Acad. Sci., USA, 95:3003-3007, 1998, and Yoshida, M. et al., J. Biol. Chem., 265:17174-17179, 1990.

For example, an enzymatic assay to determine the activity of a histone deacetylase inhibitor compound can be conducted as follows. Briefly, the effect of an HDAC inhibitor compound on affinity purified human epitope-tagged (Flag) HDAC1 can be assayed by incubating the enzyme preparation in the absence of substrate on ice for about 20 minutes with the indicated amount of inhibitor compound. Substrate ([3H]acetyl-labelled murine erythroleukemia cell-derived histone) can be added and the sample can be incubated for 20 minutes at 37° C. in a total volume of 30 mL. The reaction can then be stopped and released acetate can be extracted and the amount of radioactivity release determined by scintillation counting. An alternative assay useful for determining the activity of a histone deacetylase inhibitor compound is the “HDAC Fluorescent Activity Assay; Drug Discovery Kit-AK-500” available from BIOMOL® Research Laboratories, Inc., Plymouth Meeting, Pa.

In vivo studies can be conducted as follows. Animals, for example mice, can be injected intraperitoneally with an HDAC inhibitor compound. Selected tissues, for example brain, spleen, liver etc, can be isolated at predetermined times, post administration. Histones can be isolated from tissues essentially as described by Yoshida et al., J. Biol. Chem. 265:17174-17179, 1990. Equal amounts of histones (about 1 mg) can be electrophoresed on 15% SDS-polyacrylamide gels and can be transferred to Hybond-P filters (available from Amersham). Filters can be blocked with 3% milk and can be probed with a rabbit purified polyclonal anti-acetylated histone H4 antibody (αAc-H4) and anti-acetylated histone H3 antibody (αAc-H3) (Upstate Biotechnology, Inc.). Levels of acetylated histone can be visualized using a horseradish peroxidase-conjugated goat anti-rabbit antibody (1:5000) and the SuperSignal chemiluminescent substrate (Pierce). As a loading control for the histone protein, parallel gels can be run and stained with Coomassie Blue (CB).

In addition, hydroxamic acid-based HDAC inhibitors have been shown to up regulate the expression of the p21^{WAF1} gene. The p21^{WAF1} protein is induced within 2 hours of culture with HDAC inhibitors in a variety of transformed cells using standard methods. The induction of the p21^{WAF1} gene is associated with accumulation of acetylated histones in the chromatin region of this gene. Induction of p21WAF1 can therefore be recognized as involved in the G1 cell cycle arrest caused by HDAC inhibitors in transformed cells.

Typically, HDAC inhibitors fall into five general classes: 1) hydroxamic acid derivatives; 2) Short-Chain Fatty Acids (SCFAs); 3) cyclic tetrapeptides; 4) benzamides; and 5) electrophilic ketones.

Thus, the present invention includes within its broad scope the use of HDAC inhibitors which are 1) hydroxamic acid derivatives; 2) Short-Chain Fatty Acids (SCFAs); 3) cyclic tetrapeptides; 4) benzamides; 5) electrophilic ketones; and/or any other class of compounds capable of inhibiting histone deacetylases, for the prevention and/or treatment of TRX-mediated diseases.

Examples of such HDAC inhibitors include, but are not limited to:

A. Hydroxamic acid derivatives such as Suberoylanilide Hydroxamic Acid (SAHA) (Richon et al., Proc. Natl. Acad. Sci. USA 95, 3003-3007 (1998)); M-Carboxycinnamic Acid Bishydroxamide (CBHA) (Richon et al., supra); pyroxamide; CBHA; Trichostatin analogues such as Trichostatin A (TSA) and Trichostatin C (Koghe et al. 1998. Biochem. Pharmacol. 56: 1359-1364); Salicylihydroxamic Acid (SBHA) (Andrews et al., International J. Parasitology 30, 761-768 (2000)); Azelaic Bishydroxamic Acid (ABHA) (Andrews et al., supra); Azelaic-1-Hydroxamate-9-Anilide (AAHA) (Qiu et al., Mol. Biol. Cell 11, 2069-2083 (2000)); 6-(3-Chlorophenylureido) carpoic Hydroxamic Acid (3C1-UCHA), Oxamflatin [(2E)-5-[3-[(phenylsuibnyl)amino phenyl]-pent-2-en-4-ynohydroxamic acid (Kim et al. Oncogene, 18: 2461 2470 (1999)); A-161906, Scriptaid (Su et al. 2000 Cancer Research, 60: 3137-3142); PXD-101 (Prolifix); LAQ-824; CHAP; MW2796 (Andrews et al., supra); and MW2996 (Andrews et al., supra).

B. Cyclic tetrapeptides such as Trapoxin A (TPX)-Cyclic Tetrapeptide (cyclo-(L-phenylalanyl-L-phenylalanyl-D-pipecolinyl-L-2-amino-8-oxo-9,10-epoxy decanoyl)) (Kijima et al., J Biol. Chem. 268, 22429-22435 (1993)); FR901228 (FK 228, Depsipeptide) (Nakajima et al., Ex. Cell Res. 241, 126-133 (1998)); FR225497 Cyclic Tetrapeptide (H. Mori et al., PCT Application WO 00/08048 (17 Feb. 2000)); Apicidin Cyclic Tetrapeptide [cyclo (N O-methyl-L-tryptophanyl-L-isoleucinyl-D-pipecolinyl-L-2-amino-8oxodecanoyl)] (Darkin-Rattray et al., Proc. Natl. Acad. Sci. USA 93, 13143-13147 (1996)); Apicidin Ia, Apicidin Ib, Apicidin Ic, Apicidin IIa, and Apicidin IIb (P. Dulski et al., PCT Application WO 97/11366); CHAP, HC-Toxin Cyclic Tetrapeptide (Bosch et al., Plant Cell 7, 1941-1950 (1995)); WF27082 Cyclic Tetrapeptide (PCT Application WO 98/48825); and Chlamydocin (Bosch et al., supra).

C. Short chain fatty acid (SCFA) derivatives such as: Sodium Butyrate (Cousens et al., J. Biol. Chem. 254, 1716-1723 (1979)); Isovalerate (McBain et al., Biochem. Pharm. 53: 1357-1368 (1997)); Valerate (McBain et al., supra); 4 Phenylbutyrate (4-PBA) (Lea and Tulsyan, Anticancer Research, 15, 879-873 (1995)); Phenylbutyrate (PB) (Wang et al., Cancer Research, 59, 2766-2799 (1999)); Propionate (McBain et al., supra); Butyramide (Lea and Tulsyan, supra); Isobutyramide (Lea and Tulsyan, supra); Phenylacetate (Lea and Tulsyan, supra); 3-Bromopropionate (Lea and Tulsyan, supra); Tributyrin (Guan et al., Cancer Research, 60, 749-755 (2000)); Valproic acid and Valproate.

D. Benzamide derivatives such as CI-994; MS-27-275 [N-(2-aminophenyl)-4-[N-(pyridin-3-yl methoxycarbonyl) aminomethyl]benzamide] (Saito et al., Proc. Natl. Acad. Sci. USA 96, 4592-4597 (1999)); and 3′-amino derivative of MS-27-275 (Saito et al., supra).

E. Electrophilic ketone derivatives such as trifluoromethyl ketones (Frey et al., Bioorganic & Med. Chem. Lett. (2002), 12, 3443-3447; U.S. Pat. No. 6,511,990) and a-keto amides such as N-methyl-a-ketoamides

F. Other HDAC Inhibitors such as Depudecin (Kwon et al. 1998. PNAS 95: 3356-3361.

Preferred hydroxamic acid based HDAC inhibitor are suberoylanilide hydroxamic acid (SAHA), m-carboxycinnamic acid bishydroxamate (CBHA) and pyroxamide or pharmaceutically acceptable salts or hydrates thereof. SAHA has been shown to bind directly in the catalytic pocket of the histone deacetylase enzyme. SAHA induces cell cycle arrest, differentiation and/or apoptosis of transformed cells in culture and inhibits tumor growth in rodents. SAHA is effective at inducing these effects in both solid tumors and hematological cancers. It has been shown that SAHA is effective at inhibiting tumor growth in animals with no toxicity to the animal. The SAHA-induced inhibition of tumor growth is associated with an accumulation of acetylated histones in the tumor. SAHA is effective at inhibiting the development and continued growth of carcinogen-induced (N-methylnitrosourea) mammary tumors in rats. SAHA was administered to the rats in their diet over the 130 days of the study. Thus, SAHA is a nontoxic, orally active antitumor agent whose mechanism of action involves the inhibition of histone deacetylase activity.

SAHA can be represented by the following structural formula:

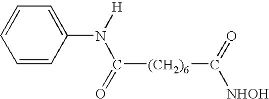

Pyroxamide can be represented by the following structural formula:

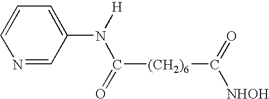

CBHA can be represented by the structural formula:

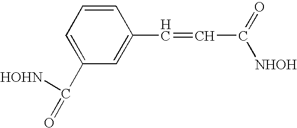

In one embodiment, the HDAC inhibitor can be represented by Formula I: 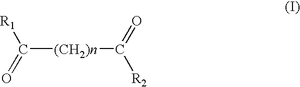
wherein R₁ and R₂ can be the same or different; when R₁ and R₂ are the same, each is a substituted or unsubstituted arylamino, cycloalkylamino, pyridineamino, piperidino, 9-purine-6-amine or thiazoleamino group; when R₁ and R₂ are different R₁═R₃—N—R₄, wherein each of R₃ and R₄ are independently the same as or different from each other and are a hydrogen atom, a hydroxyl group, a substituted or unsubstituted, branched or unbranched alkyl, alkenyl, cycloalkyl, aryl alkyloxy, aryloxy, arylalkyloxy or pyridine group, or R₃ and R₄ are bonded together to form a piperidine group, R₂ is a hydroxylamino, hydroxyl, amino, alkylamino, dialkylamino or alkyloxy group and n is an integer from about 4 to about 8 or pharmaceutically acceptable salts or hydrates thereof.

As such, in another embodiment the HDAC inhibitors used in the method of the invention can be represented by Formula II: 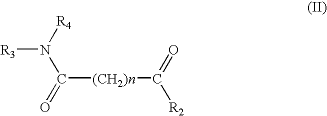
wherein each of R₃ and R₄ are independently the same as or different from each other and are a hydrogen atom, a hydroxyl group, a substituted or unsubstituted, branched or unbranched alkyl, alkenyl, cycloalkyl, arylalkyloxy, aryloxy, arylalkyloxy or pyridine group, or R₃ and R₄ are bonded together to form a piperidine group, R₂ is a hydroxylamino, hydroxyl, amino, alkylamino, dialkylamino or alkyloxy group and n is an integer from about 4 to about 8 or pharmaceutically acceptable salts or hydrates thereof.

In a particular embodiment of Formula II, R₂ is a hydroxylamino, hydroxyl, amino, methylamino, dimethylamino or methyloxy group and n is 6. In yet another embodiment of Formula II, R₄ is a hydrogen atom, R₃ is a substituted or unsubstituted phenyl and n is 6. In further embodiments of Formula II, R₄ is hydrogen and R₃ is an a-, β-, or γ-pyridine.

In other specific embodiments of Formula II, R₄ is a hydrogen atom and R₃ is a cyclohexyl group; R₄ is a hydrogen atom and R₃ is a methoxy group; R₃ and R₄ each bond together to form a piperidine group; R₄ is a hydrogen atom and R₃ is a hydroxyl group; R₃ and R₄ are both a methyl group and R₃ is phenyl and R₄ is methyl.

Further HDAC inhibitors suitable for use in the present invention can be represented by structural Formula III: 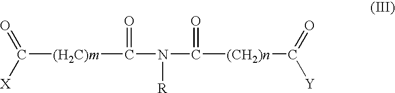
wherein each of X and Y are independently the same as or different from each other and are a hydroxyl, amino or hydroxylamino group, a substituted or unsubstituted alkyloxy, alkylamino, dialkylamino, arylamino, alkylarylamino, alkyloxyamino, aryloxyamino, alkyloxyalkylamino, or aryloxyalkyl amino group; R is a hydrogen atom, a hydroxyl, group, a substituted or unsubstituted alkyl, arylalkyloxy, or aryloxy group; and each of m and n are independently the same as or different from each other and are each an integer from about 0 to about 8 or pharmaceutically acceptable salts or hydrates thereof.

In a particular embodiment, the HDAC inhibitor is a compound of Formula III wherein X, Y and R are each hydroxyl and both m and n are 5. In yet another embodiment, the UDAC inhibitor compounds suitable for use in the method of the invention can be represented by structural Formula IV:

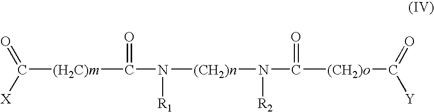

wherein each of X and Y are independently the same as or different from each other and are a hydroxyl, amino or hydroxylamino group, a substituted or unsubstituted alkyloxy, alkylamino, dialkylamino, arylamino, alkylarylamino, alkyloxyamino, aryloxyamino, alkyloxyalkylamino or aryloxyalkylamino group; each of R₁ and R₂ are independently the same as or different from each other and are a hydrogen atom, a hydroxyl group, a substituted or unsubstituted alkyl, aryl, alkyloxy, or aryloxy group; and each of m, n and o are independently the same as or different from each other and are each an integer from about 0 to about 8 or pharmaceutically acceptable salts or hydrates thereof.

Other HDAC inhibitors suitable for use in the invention include compounds having structural Formula V:

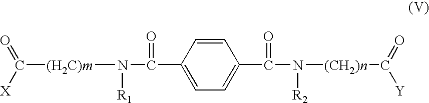

wherein each of X and Y are independently the same as or different from each other and are a hydroxyl, amino or hydroxylamino group, a substituted or unsubstituted alkyloxy, alkylamino, dialkylamino, arylamino, alkylarylamino, alkyloxyamino, aryloxyamino, alkyloxyalkylamino or aryloxyalkylamino group; each of R₁ and R₂ are independently the same as or different from each other and are a hydrogen atom, a hydroxyl group, a substituted or unsubstituted alkyl, aryl, alkyloxy, or aryloxy group; and each of m and n are independently the same as or different from each other and are each an integer from about 0 to about 8 or pharmaceutically acceptable salts or hydrates thereof.

In a further embodiment, HDAC inhibitors suitable for use in the method of the present invention can have structural Formula VI:

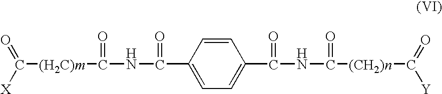

wherein each of X and Y are independently the same as or different from each other and are a hydroxyl, amino or hydroxylamino group, a substituted or unsubstituted alkyloxy, alkylamino, dialkylamino, arylamino, alkylarylamino, alkyloxyamino, aryloxyamino, alkyloxyalkylamino or aryloxyalkylamino group; and each of m and n are independently the same as or different from each other and are each an integer from about 0 to about 8 or pharmaceutically acceptable salts or hydrates thereof.

In yet another embodiment, the HDAC inhibitors useful in the method of the invention can have structural Formula VII: 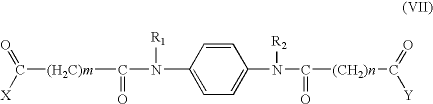
wherein each of X and Y are independently the same as or different from each other and are a hydroxyl, amino or hydroxylamino group, a substituted or unsubstituted alkyloxy, alkylamino, dialkylamino, arylamino, alkylarylamino, alkyloxyamino, aryloxyamino, alkyloxyalkylamino or aryloxyalkylamino group; R₁ and R₂ are independently the same as or different from each other and are a hydrogen atom, a hydroxyl group, a substituted or unsubstituted alkyl, arylalkyloxy or aryloxy group; and each of m and n are independently the same as or different from each other and are each an integer from about 0 to about 8 or pharmaceutically acceptable salts or hydrates thereof.

In yet a further embodiment, HDAC inhibitors suitable for use in the invention can have structural Formula VIII:

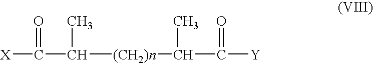

wherein each of X an Y are independently the same as or different from each other and are a hydroxyl, amino or hydroxylamino group, a substituted or unsubstituted alkyloxy, alkylamino, dialkylamino, arylamino, alkylarylamino, or aryloxyalkylamino group; and n is an integer from about 0 to about 8 or pharmaceutically acceptable salts or hydrates thereof.

Additional compounds suitable for use in the method of the invention include those represented by Formula IX:

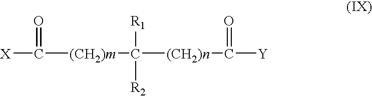

wherein Each of X and Y are independently the same as or different from each other and are a hydroxyl, amino or hydroxylamino group, a substituted or unsubstituted alkyloxy, alkylamino, dialkylamino, arylamino, alkylarylamino, alkyloxyamino, aryloxyamino, alkyloxyalkylamino or aryloxyalkylamino group; each of R₁ and R₂ are independently the same as or different from each other and are a hydrogen atom, a hydroxyl group, a substituted or unsubstituted alkyl, aryl, alkyloxy, aryloxy, carbonylhydroxylamino or fluoro group; and each of m and n are independently the same as or different from each other and are each an integer from about 0 to about 8 or pharmaceutically acceptable salts and hydrates thereof.

In a further embodiment, HDAC inhibitors suitable for use in the invention include compounds having structural Formula X:

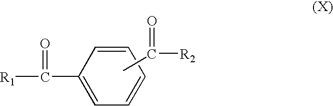

wherein each of R₁ and R₂ are independently the same as or different from each other and are a hydroxyl, alkyloxy, amino, hydroxylamino, alkylamino, dialkylamino, arylamino, alkylarylamino, alkyloxyamino, aryloxyamino, alkyloxyalkylamino, or aryloxyalkylamino group. In a particular embodiment, the HDAC inhibitor is a compound of structural Formula X wherein R₁ and R₂ are both hydroxylamino or pharmaceutically acceptable salts or hydrates thereof.

In a further embodiment, the HDAC inhibitor suitable for use in the invention has structural Formula XI: 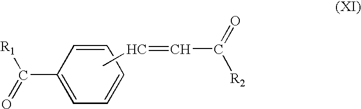
wherein each of R₁ and R₂ are independently the same as or different from each other and are a hydroxyl, alkyloxy, amino, hydroxylamino, alkylamino, dialkylamino, arylamino, alkylarylamino, alkyloxyamino, aryloxyamino, alkyloxyalkylamino, or aryloxyalkylamino group or pharmaceutically acceptable salts or hydrates thereof. In a particular embodiment, the HDAC inhibitor is a compound of structural Formula XI wherein R₁ and R₂ are both hydroxylamino.

In a further embodiment, HDAC inhibitors suitable for use in the present invention include compounds represented by structural Formula XII:

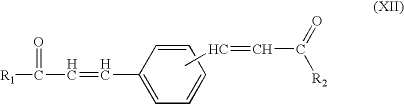

wherein each of R₁ and R₂ are independently the same as or different from each other and are a hydroxyl, alkyloxy, amino, hydroxylamino, alkylamino, dialkylamino, arylamino, alkylarylamino, alkyloxyamino, aryloxyamino, alkyloxyalkylamino, or aryloxyalkylamino group or pharmaceutically acceptable salts or hydrates thereof. In a particular embodiment, the HDAC inhibitor is a compound of structural Formula XII wherein R₁ and R₂ are both hydroxylamino.

Additional compounds suitable for use in the method of the invention include those represented by structural Formula XIII: 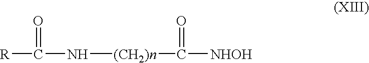
wherein R is a substituted or unsubstituted phenyl, piperidine, thiazole, 2-pyridine, 3-pyridine or 4-pyridine and n is an integer from about 4 to about 8 or pharmaceutically acceptable salts or hydrates thereof.

In yet another embodiment, the HDAC inhibitors suitable for use in the method of the invention can be represented by structural Formula (XIV): 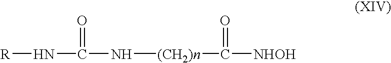
wherein R is a substituted or unsubstituted phenyl, pyridine, piperidine or thiazole group and n is an integer from about 4 to about 8 or pharmaceutically acceptable salts or hydrates thereof.

In a particular embodiment, R is phenyl and n is 5. In another embodiment, n is 5 and R is 3-chlorophenyl.

In structural formulas I-XIV, substituted phenyl, refers to a phenyl group which can be substituted with, for example, but not limited to a methyl, cyano, nitro, trifluoromethyl, amino, aminocarbonyl, methylcyano, halogen, e.g., chloro, fluoro, bromo, iodo, 2,3-difluoro, 2,4-difluoro, 2,5-difluoro, 3,4-difluoro, 3,5-difluoro, 2,6-difluoro, 1,2,3-trifluoro, 2,3,6-trifluoro, 2,3,4,5,6-pentafluoro, azido, hexyl, t-butyl, phenyl, carboxyl, hydroxyl, methyloxy, benzyloxy, phenyloxy, phenylaminooxy, phenylaminocarbonyl, methyloxycarbonyl, methylaminocarbonyl, dimethylamino, dimethylaminocarbonyl or hydroxyaminocarbonyl group.

Other HDAC inhibitors useful in the present invention can be represented by structural Formula XV: 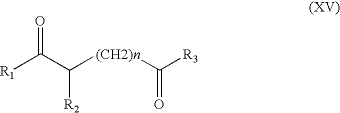
wherein each of R₁ and R₂ is directly attached or through a linker and is substituted or unsubstituted, aryl (e.g. naphthyl, phenyl), cycloalkyl, cycloalkylamino, pyridineamino, piperidino, 9-purine-6-amine, thiazoleamino group, hydroxyl, branched or unbranched alkyl, alkenyl, alkyloxy, aryloxy, arylalkyloxy, or pyridine group; n is an integer from about 3 to about 10 and R₃ is a hydroxamic acid, hydroxylamino, hydroxyl, amino, alkylamino or alkyloxy group or pharmaceutically acceptable salts or hydrates thereof.

The linker can be an amide moiety, —O—, —S—, —NH— or —CH2—.

In certain embodiments, R₁ is —NH—R₄ wherein R₄ is substituted or unsubstituted, aryl (e.g., naphthyl, phenyl), cycloalkyl, cycloalkylamino, pyridineamino, piperidino, 9-purine-6-amine, thiazoleamino group, hydroxyl, branched or unbranched alkyl, alkenyl, alkyloxy, aryloxy, arylalkyloxy or pyridine group.

Further and more specific HDAC inhibitors of Formula XV, include those which can be represented by Formula XVI: 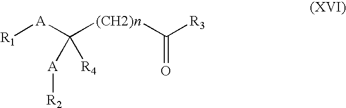
wherein each of R₁ and R₂ is, substituted or unsubstituted, aryl (e.g., phenyl, naphthyl), cycloalkyl, cycloalkylamino, pyridineamino, piperidino, 9-purine-6-amine, thiazoleamino group, hydroxyl, branched or unbranched alkyl, alkenyl, alkyloxy, aryloxy, arylalkyloxy or pyridine group; R₃ is hydroxamic acid, hydroxylamino, hydroxyl, amino, alkylamino or alkyloxy group; R₄ is hydrogen, halogen, phenyl or a cycloalkyl moiety; and A can be the same or different and represents an amide moiety, —O—, —S—, —NR₅— or —CH₂— where R₁ is a substitute or unsubstituted C₁-C₅ alkyl and n is an integer from 3 to 10 or pharmaceutically acceptable salts or hydrates thereof.

For example, further compounds having a more specific structure within Formula XVI can be represented by structural Formula XVII: 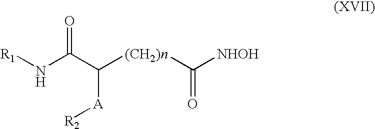
wherein A is an amide moiety, R₁ and R₂ are each selected from substituted or unsubstituted aryl (e.g., phenyl, naphthyl), pyridineamino, 9-purine-6-amine, thiazoleamino, aryloxy, arylalkyloxy or pyridine and n is an integer from 3 to 10 or pharmaceutically acceptable salts or hydrates thereof.

For example, the compound can have the formula

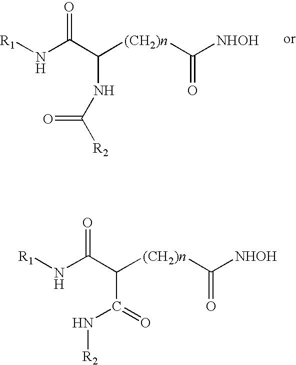

In another embodiment, the HDAC inhibitor can have the Formula XVIII: 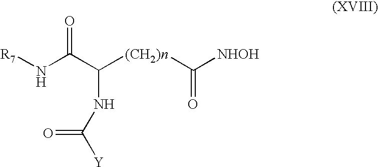
wherein R₇ is selected from substituted of unsubstituted aryl (e.g., phenyl or naphthyl), pyridineamino, 9-purine-6-amine, thiazoleamino, aryloxy, arylalkyloxy or pyridine and n is an integer from 3 to 10 and Y is selected from 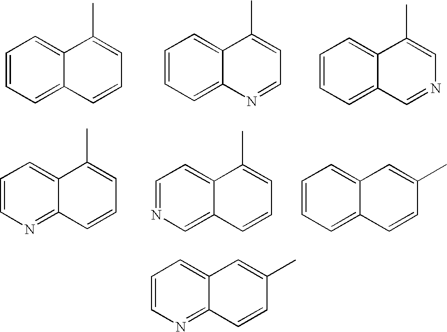
or pharmaceutically acceptable salts or hydrates thereof.

In a further embodiment, the HDAC inhibitor compound can have Formula XIX: 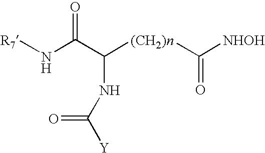
wherein n is an integer from 3 to 10, Y is selected from 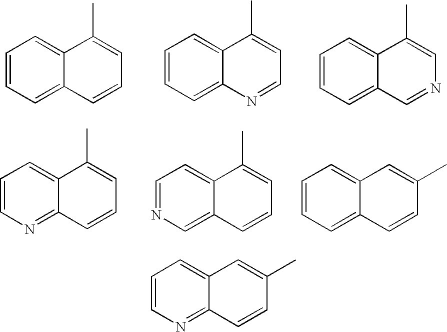
and R₇′ is selected from 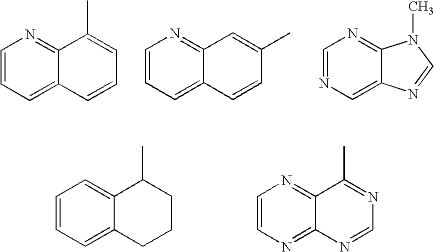
or pharmaceutically acceptable salts or hydrates thereof.

Further compounds for use in the invention can be represented by structural Formula XX: 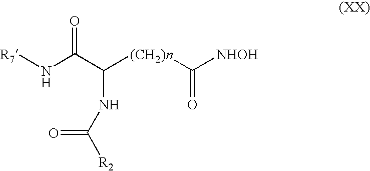
wherein R₂ is selected from substituted or unsubstituted aryl, substituted or unsubstituted naphtha, pyridineamino, 9-purine-6-amine, thiazoleamino, substituted or unsubstituted aryloxy, substituted or unsubstituted arylalkyloxy or pyridine and n is an integer from 3 to 10 and R₇′ is selected from 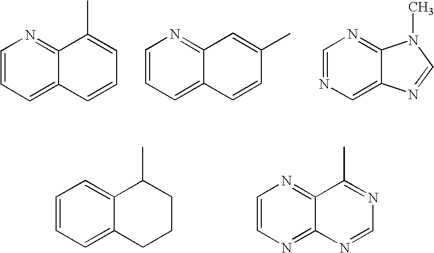
or pharmaceutically acceptable salts or hydrates thereof.

Further HDAC inhibitors useful in the invention can be represented by structural Formula XXI: 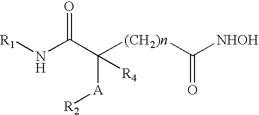
wherein A is an amide moiety, R₁ and R₂ are each selected from substituted or unsubstituted aryl, naphtha, pyridineamino, 9-purine-6-amine, thiazoleamino, aryloxy, arylalkyloxy or pyridine, R₄ is hydrogen, a halogen, a phenyl or a cycloalkyl moiety and n is an integer from 3 to 10 or pharmaceutically acceptable salts or hydrates thereof.

For example, a compound of Formula XXI can be represented by the structure: 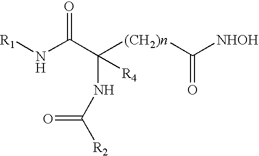
or can be represented by the structure: 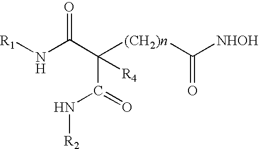
wherein R₁, R₂, R₄ and n have the meanings of Formula XXI or pharmaceutically acceptable salts or hydrates thereof.

Further, HDAC inhibitors having the structural Formula XXII: 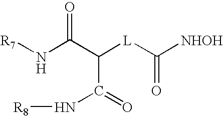
wherein L is a linker selected from the group consisting of —(CH₂)n-, —(CH═CH)m, phenyl, -cycloalkyl-, or any combination thereof; and wherein each of R₇ and R₈ are independently substituted or unsubstituted, aryl, naphtha, pyridineamino, 9-purine-6-amine, thiazoleamino group, aryloxy, arylalkyloxy, or pyridine group, n is an integer from 3 to 10 and m is an integer from 0-10 or pharmaceutically acceptable salts or hydrates thereof.

For example, a compound of Formula XXII can be:

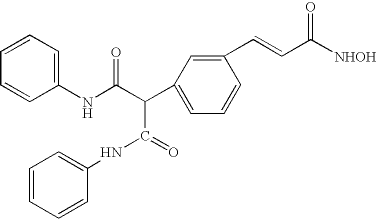

Other HDAC inhibitors suitable for use in the invention include those shown in the following more specific formulas: 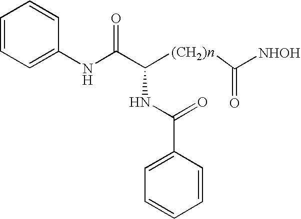
wherein n is an integer from 3 to 10 or an enantiomer, or 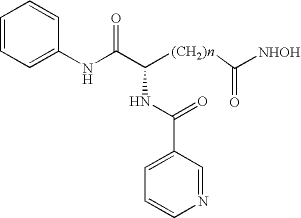
wherein n is an integer from 3 to 10 or an enantiomer, or 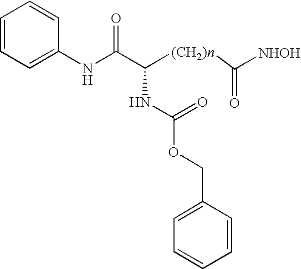
wherein n is an integer from 3 to 10 or an enantiomer, or 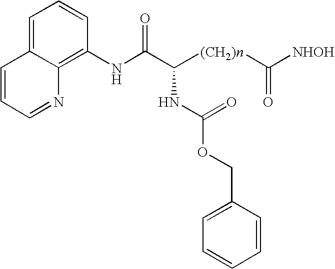
wherein n is an integer from 3 to 10 or an enantiomer, or 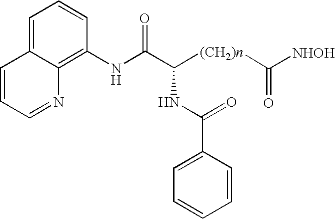
wherein n is an integer from 3 to 10 or an enantiomer or pharmaceutically acceptable salts or hydrates of all of the above.

Further specific HDAC inhibitors suitable for use in the invention include 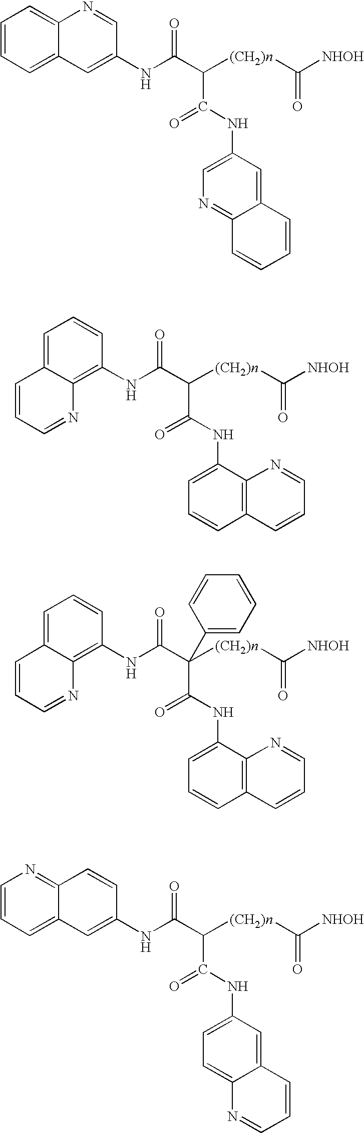
wherein n in each is an integer from 3 to 10 or pharmaceutically acceptable salts or hydrates of all of the above, and the compound

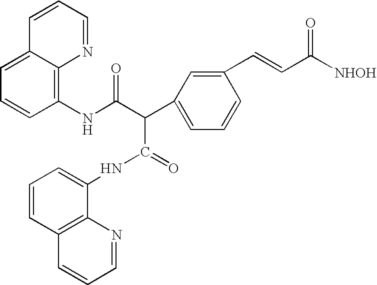

Other examples of such compounds and other HDAC inhibitors can be found in U.S. Pat. No. 5,369,108, issued on Nov. 29, 1994, U.S. Pat. No. 5,700,811, issued on Dec. 23, 1997, U.S. Pat. No. 5,773,474, issued on Jun. 30, 1998, U.S. Pat. No. 5,932,616 issued on Aug. 3, 1999 and U.S. Pat. No. 6,511,990, issued Jan. 28, 2003 all to Breslow et al.; U.S. Pat. No. 5,055,608, issued on Oct. 8, 1991, U.S. Pat. No. 5,175,191, issued on Dec. 29, 1992 and U.S. Pat. No. 5,608,108, issued on Mar. 4, 1997 all to Marks et al.; as well as, Yoshida, M., et al., Bioassays 17, 423-430 (1995); Saito, A., et al., PNAS USA 96, 4592-4597, (1999); Furamai R. et al., PNAS USA 98 (1), 87-92 (2001); Kornatsu, Y., et al., Cancer Res. 61(11), 4459-4466(2001); Su, G. H., et al., Cancer Res. 60, 3137-3142(2000); Lee, B. I. et al., Cancer Res. 61(3), 931-934; Suzuki, T., et al., J. Med. Chem. 42(15), 3001-3003 (1999); published PCT Application WO 01/18171 published on Mar. 15, 2001 to Sloan-Kettering Institute for Cancer Research and The Trustees of Columbia University; published PCT Application WO02/246144 to Hoffmann-La Roche; published PCT Application WO02/22577 to Novartis; published PCT Application WO02/30879 to Prolifix; published PCT Applications WO 01/38322 (published May 31, 2001), WO 01/70675 (published on Sep. 27, 2001) and WO 00/71703 (published on Nov. 30, 2000) all to Methylgene, Inc.; published PCT Application WO 00/21979 published on Oct. 8, 1999 to Fujisawa Pharmaceutical Co., Ltd.; published PCT Application WO 98/40080 published on Mar. 11, 1998 to Beacon Laboratories, L.L.C.; and Curtin M. (Current patent status of histone deacetylase inhibitors Expert Opin. Ther. Patents (2002) 12(9): 1375-1384 and references cited therein).

Specific non-limiting examples of HDAC inhibitors are provided in the Table below. It should be noted that the present invention encompasses any compounds which are structurally similar to the compounds represented below, and which are capable of inhibiting histone deacetylases.

Compound Name

| | |
|---|---|
| MS-275 | 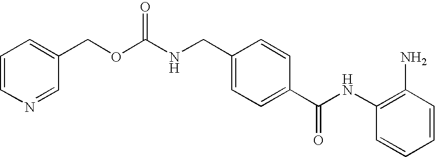 |
| DEPSIPEPTIDE | 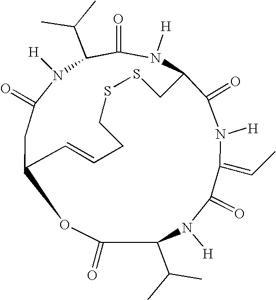 |
| CI-994 | 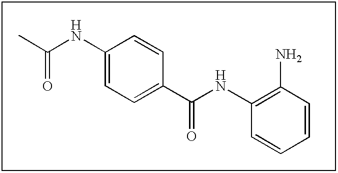 |
| APICIDIN | 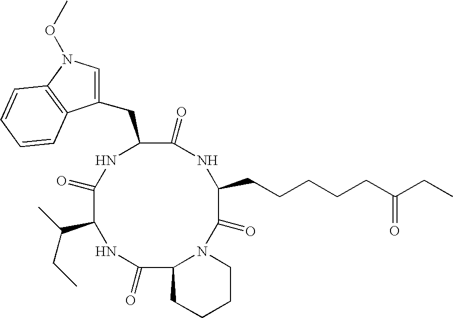 |
| A-161906 | 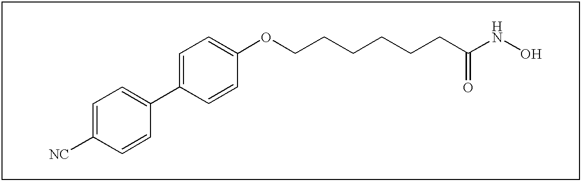 |
| SCRIPTAID | 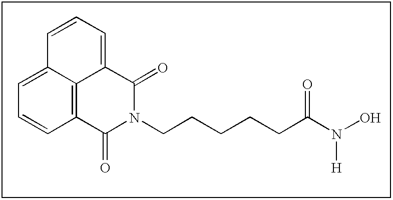 |
| PXD-101 | 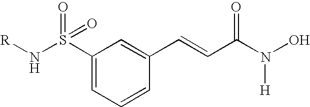 |
| CHAP | 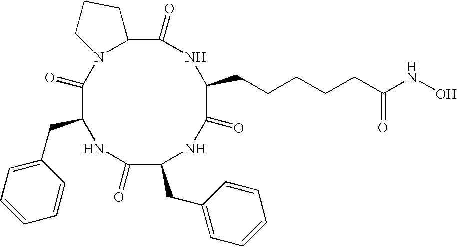 |
| LAQ-824 | 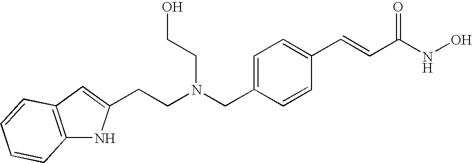 |
| BUTYRIC ACID | 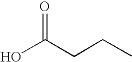 |
| DEPUDECIN | 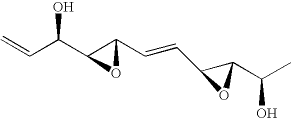 |
| OXAMFLATIN | 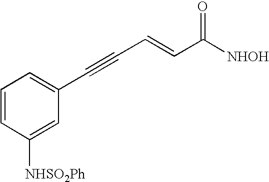 |
| TRICHOSTATIN C | 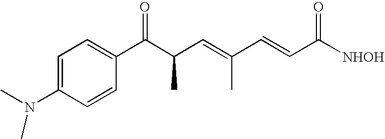 |

The active compounds disclosed can, as noted above, be prepared in the form of their pharmaceutically acceptable salts. Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; (b) salts formed from elemental anions such as chlorine, bromine, and iodine, and (c) salts derived from bases, such as ammonium salts, alkali metal salts such as those of sodium and potassium, alkaline earth metal salts such as those of calcium and magnesium, and salts with organic bases such as dicyclohexylamine and N-methyl-D-glucamine.

The active compounds disclosed can, as noted above, be prepared in the form of their hydrates, such as hemihydrate, monohydrate, dihydrate, trihydrate, tetrahydrate and the like.

“Therapeutically effective amount” as that term is used herein refers to an amount which regulates, for example, increases, decreases or maintains a physiologically suitable level of TRX in the patient in need of treatment to elicit the desired therapeutic effect. The therapeutic effect is dependent upon the disease being treated. As such, the therapeutic effect can be a decrease in the severity of symptoms associated with the disease and/or inhibition (partial or complete) of progression of the disease. The amount needed to elicit the therapeutic response can be determined based on the age, health, size and sex of the patient. Optimal amounts can also be determined based on monitoring of the patient's response to treatment, for example, determination of the TRX levels in the synovial fluid and/or synovial tissue of a patient suffering from rheumatoid arthritis.

“Patient” or “subject” as that term is used herein, refers to the recipient of the treatment. Mammalian and non-mammalian patients are included. In a specific embodiment, the patient is a mammal, such as a human, canine, murine, feline, bovine, ovine, swine or caprine. In a preferred embodiment, the patient is a human.

Thioredoxin (TRX)-Mediated Diseases

As defined herein, a disease or medical condition is considered to be a “TRX-mediated disease” if the spontaneous or experimental disease or medical condition is associated with abnormal levels, for example, elevated or suppressed levels of TRX in bodily fluids or tissue or if cells or tissues taken from the body produce abnormal levels of TRX in culture. In many cases, such TRX-mediated diseases can also be recognized by the following additional two conditions: (1) pathological findings associated with the disease or medical condition can be mimicked experimentally in animals by the administration or sequestration of TRX; and (2) the pathology induced in experimental animal models of the disease or medical condition can be inhibited or abolished by treatment with agents which increase, decrease or maintain the action of TRX depending on the disease or medical condition. In most TRX-mediated diseases at least two of the three conditions can be met, and in many TRX-mediated diseases all three conditions can be met.

As contemplated herein, the HDAC inhibitors of the present invention are effective at treating TRX-mediated diseases which are characterized by abnormal levels of TRX in bodily fluids/tissue or in a culture of cells taken from the body of a subject afflicted with a TRX-mediated disease. An “abnormal level” refers to elevated or suppressed levels of TRX, compared to a level of TRX in the bodily fluids/tissue of a subject who is not afflicted with a TRX-mediated disease. The level of TRX refers in one embodiment to the level of expression of TRX, for example the amount of protein that is expressed or the amount of gene (m-RNA) that is expressed. In another embodiment, the level of TRX refers to the enzymatic activity of TRX or TRX-associated proteins such as thioredoxin reductase (TR), for example elevated or suppressed levels of TRX or TRX-TR enzymatic activity.

A non-exclusive list of acute and chronic diseases which can be TRX-mediated diseases include but are not limited to inflammatory diseases, autoimmune diseases, allergic diseases, diseases associated with oxidative stress, and diseases characterized by cellular hyperproliferation. Non-limiting examples are inflammatory conditions of a joint including and rheumatoid arthritis (RA) and psoriatic arthritis; inflammatory bowel diseases such as Crohn's disease and ulcerative colitis; spondyloarthropathies; scleroderma; psoriasis (including T-cell mediated psoriasis) and inflammatory dermatoses such an dermatitis, eczema, atopic dermatitis, allergic contact dermatitis, urticaria; vasculitis (e.g., necrotizing, cutaneous, and hypersensitivity vasculitis); eosinphilic myositis, eosinophilic fasciitis; cancers with leukocyte infiltration of the skin or organs, ischemic injury, including cerebral ischemia (e.g., brain injury as a result of trauma, epilepsy, hemorrhage or stroke, each of which can lead to neurodegeneration); HIV, heart failure, chronic, acute or malignant liver disease, autoimmune thyroiditis; systemic lupus erythematosus, Sjorgren's syndrome, lung diseases (e.g., ARDS); acute pancreatitis; amyotrophic lateral sclerosis (ALS); Alzheimer's disease; cachexia/anorexia; asthma; atherosclerosis; chronic fatigue syndrome, fever; diabetes (e.g., insulin diabetes or juvenile onset diabetes); glomerulonephritis; graft versus host rejection (e.g., in transplantation); hemohorragic shock; hyperalgesia: inflammatory bowel disease; multiple sclerosis; myopathies (e.g., muscle protein metabolism, esp. in sepsis); osteoporosis; Parkinson's disease; pain; pre-term labor; psoriasis; reperfusion injury; cytokine-induced toxicity (e.g., septic shock, endotoxic shock); side effects from radiation therapy, temporal mandibular joint disease, tumor metastasis; or an inflammatory condition resulting from strain, sprain, cartilage damage, trauma such as bum, orthopedic surgery, infection or other disease processes. Allergic diseases and conditions, include but are not limited to respiratory allergic diseases such as asthma, allergic rhinitis, hypersensitivity lung diseases, hypersensitivity pneumonitis, eosinophilic pneumonias (e.g., Loeffler's syndrome, chronic eosinophilic pneumonia), delayed-type hypersentitivity, interstitial lung diseases (ILD) (e.g., idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis); systemic anaphylaxis or hypersensitivity responses, drug allergies (e.g., to penicillin, cephalosporins), insect sting allergies, and the like.

In one embodiment, the TRX-mediated disease is an inflammatory condition of the joint, for example rheumatoid arthritis. Inflammatory conditions of a joint are chronic joint diseases that afflict and disable, to varying degrees, millions of people worldwide. RA is a TRX-mediated disease of articular joints in which the cartilage and bone are slowly eroded away by a proliferative, invasive connective tissue called pannus, which is derived from the synovial membrane. The disease can involve peri-articular structures such as bursae, tendon sheaths and tendons as well as extra-articular tissues such as the subcutis, cardiovascular system, lungs, spleen, lymph nodes, skeletal muscles, nervous system (central and peripheral) and eyes (Silberberg (1985), Anderson's Pathology, Kissane (ed.), II:1828).

In RA the synovial tissue is infiltrated with mononuclear cells, including macrophages and T cells, and to a lesser extent B cells and dendritic cells which are believed to play a crucial role in the pathogenesis of RA. Maurice et al. (Arthritis and Rheumatism, 40:2430-2439, 1999) found significantly increased TRX levels in the synovial fluid (SF) from 22 patients with RA, when compared with plasma levels in the same patients (P<0.001).

In a particular embodiment, the method of invention is a method of treating rheumatoid arthritis is a patient in need thereof comprising administering to said patient a therapeutically effective amount of a histone deacetylase inhibitor. In a particularly preferred embodiment, the method of treating rheumatoid arthritis in a patient in need thereof comprises administering a therapeutically effective amount of suberoylanilide hydroxamic acid. In another preferred embodiment, the method of treating rheumatoid arthritis in a patient in need thereof comprises administering a therapeutically effective amount of pyroxamide. In another preferred embodiment, the method of treating rheumatoid arthritis in a patient in need thereof comprises administering a therapeutically effective amount of CBHA.

Without being bound by a particular theory, it is believed that TBP-2 is induced by histone deacetylase inhibitors and can bind to the reduced form of TRX resulting in a reduction in the level of this protein. The induction of the TBP-2 can be used to treat TRX-mediated inflammatory diseases in a patient by reducing the levels of TRX present in said patient. As such, administration of HDAC inhibitors to patients can result in a decrease in the levels of TRX in, for example, the synovial fluid and synovial tissue of joints when the patient is suffering from rheumatoid arthritis.

Combination Treatments

The HDAC inhibitors can be administered alone or in combination with other standard therapies for TRX-mediated diseases. In combination, as that term in used herein refers to administration of the HDAC inhibitor in combination with a therapeutically effective amount of an agent used in standard therapy for the TRX-mediated disease being treated.

For example, a therapeutically effective amount of an HDAC inhibitor can be administered in combination with a therapeutically effective amount of a COX2 inhibitor such as celecoxib to treat rheumatoid arthritis.

The pharmaceutical combinations comprising an HDAC inhibitor in combination with an agent used in standard therapy for the TRX-mediated disease being treated include administration of a single pharmaceutical dosage formulation which contains both the HDAC inhibitor and the standard therapy agent, as well as administration of each active agent in its own separate pharmaceutical dosage formulation.

Where separate dosage formulations are used, the HDAC inhibitor and the standard therapy agent can be administered at essentially the same time (concurrently) or at separately staggered times (sequentially). The pharmaceutical combination is understood to include all these regimens. Administration in these various ways are suitable for the present invention as long as the beneficial pharmaceutical effect of the HDAC inhibitor and the standard therapy agent are realized by the patient at substantially the same time. Such beneficial effect is preferably achieved when the target blood level concentrations of each active drug are maintained at substantially the same time. It is preferred that the HDAC inhibitor and the standard therapy agent be coadministered concurrently on a once-a-day dosing schedule; however, varying dosing schedules, are also encompassed herein. A single oral dosage formulation comprised of both the HDAC inhibitor and the standard therapy agent is preferred since a single dosage formulation will provide convenience for the patient.

For example, standard therapies for arthritis include analgesics such as acetaminophen; anti-inflammatory treatments such as nonsteroidal anti-inflammatory drugs (e.g. aspirin, ibuprofen, indomethacin, piroxicam); and immunosuppressive treatments such as glucocorticoids, methotrexate, cyclophosphamide, cyclosporine, azathioprine, penicillamine, hydroxychloroquine, organic gold compounds, sulfasalazine and COX2 inhibitors such as celecoxib. The HDAC inhibitor compound can therefore be administered in combination with any of the standard therapies for arthritis.

Pharmaceutical Compositions

The HDAC inhibitors of the invention can be administered in such oral forms as tablets, capsules (each of which includes sustained release or timed release formulations), pills, powders, granules, elixirs, tinctures, suspensions, syrups, and emulsions. Likewise, the HDAC inhibitors can be administered in intravenous (bolus or infusion), intraperitoneal, subcutaneous, or intramuscular form, all using forms well known to those of ordinary skill in the pharmaceutical arts.

The HDAC inhibitors can be administered in the form of a depot injection or implant preparation which can be formulated in such a manner as to permit a sustained release of the active ingredient. The active ingredient can be compressed into pellets or small cylinders and implanted subcutaneously or intramuscularly as depot injections or implants. Implants can employ inert materials such as biodegradable polymers or synthetic silicones, for example, Silastic, silicone rubber or other polymers manufactured by the Dow-Corning Corporation.

The HDAC inhibitors can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The HDAC inhibitors can also be delivered by the use of monoclonal antibodies as individual carriers to which the compound molecules are coupled. The HDAC inhibitors can also be prepared with soluble polymers as targetable drug carriers. Such polymers can include polyvinlypyrrolidone, pyran copolymer, polyhydroxy-propyl-methacrylamide-phenol, polyhydroxyethyl-aspartamide-phenol, or polyethyleneoxide-polylysine substituted with palmitoyl residues. Furthermore, the HDAC inhibitors can be prepared with biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels.

The dosage regimen utilizing the HDAC inhibitors can be selected in accordance with a variety of factors including type, species, age, weight, sex and the TRX-mediated inflammatory disease being treated; the severity of the condition to be treated; the route of administration; the renal and hepatic function of the patient; and the particular compound or salt thereof employed. An ordinarily skilled physician or veterinarian can readily determine and prescribe the effective amount of the drug required to treat, for example, to prevent, inhibit (fully or partially) or arrest the progress of the disease.

Oral dosages of the HDAC inhibitors, when used to treat the desired TRX-mediated inflammatory disease, can range between about 2 mg to about 2000 mg per day, such as from about 20 mg to about 2000 mg per day, such as from about 200 mg to about 2000 mg per day. For example, oral dosages can be about 2, about 20, about 200, about 400, about 800, about 1200, about 1600 or about 2000 mg per day. It is understood that the total amount per day can be administered in a single dose or can be administered in multiple dosings such as twice, three or four times per day.

For example, a patient can receive between about 2 mg/day to about 2000 mg/day, for example, from about 20-2000 mg/day, such as from about 200 to about 2000 mg/day, for example from about 400 mg/day to about 1200 mg/day. A suitably prepared medicament for once a day administration can thus contain between about 2 mg and about 2000 mg, such as from about 20 mg to about 2000 mg, such as from about 200 mg to about 1200 mg, such as from about 400 mg/day to about 1200 mg/day. The HDAC inhibitors can be administered in a single dose or in divided doses of two, three, or four times daily. For administration twice a day, a suitably prepared medicament would therefore contain half of the needed daily dose.

Intravenously or subcutaneously, the patient would receive the HDAC inhibitor in quantities sufficient to deliver between about 3-1500 mg/m² per day, for example, about 3, 30, 60, 90, 180, 300, 600, 900, 1200 or 1500 mg/m² per day. Such quantities can be administered in a number of suitable ways, e.g. large volumes of low concentrations of HDAC inhibitor during one extended period of time or several times a day. The quantities can be administered for one or more consecutive days, intermittent days or a combination thereof per week (7 day period). Alternatively, low volumes of high concentrations of HDAC inhibitor during a short period of time, e.g. once a day for one or more days either consecutively, intermittently or a combination thereof per week (7 day period). For example, a dose of 300 mg/m² per day can be administered for consecutive days for a total of 1500 mg/m² per treatment. In another dosing regimen, the number of consecutive days can also be, with treatment lasting for 2 or 3 consecutive weeks for a total of 3000 mg/m² and 4500 mg/m² total treatment.

Typically, an intravenous formulation can be prepared which contains a concentration of HDAC inhibitor of between about 1.0 mg/mL to about 10 mg/mL, e.g. 2.0 mg/mL, 3.0 mg/mL, 4.0 mg/mL, 5.0 mg/mL, 6.0 mg/mL, 7.0 mg/mL, 8.0 mg/mL, 9.0 mg/mL and 10 mg/mL and administered in amounts to achieve the doses described above. In one example, a sufficient volume of intravenous formulation can be administered to a patient in a day such that the total dose for the day is between about 300 and about 1500 mg/m².

Glucuronic acid, L-lactic acid, acetic acid, citric acid or any pharmaceutically acceptable acid/conjugate base with reasonable buffering capacity in the pH range acceptable for intravenous administration of the HDAC inhibitor can be used as buffers. Sodium chloride solution wherein the pH has been adjusted to the desired range with either acid or base, for example, hydrochloric acid or sodium hydroxide, can also be employed. Typically, a pH range for the intravenous formulation can be in the range of from about 5 to about 12. A preferred pH range for intravenous formulation wherein the HDAC inhibitor has a hydroxamic acid moiety, can be about 9 to about 12. Consideration should be given to the solubility and chemical compatibility of the HDAC inhibitor in choosing an appropriate excipient.

Subcutaneous formulations, preferably prepared according to procedures well known in the art at a pH in the range between about 5 and about 12, also include suitable buffers and isotonicity agents. They can be formulated to deliver a daily dose of HDAC inhibitor in one or more daily subcutaneous administrations, e.g., one, two or three times each day. The choice of appropriate buffer and pH of a formulation, depending on solubility of the HDAC inhibitor to be administered, is readily made by a person having ordinary skill in the art. Sodium chloride solution wherein the pH has been adjusted to the desired range with either acid or base, for example, hydrochloric acid or sodium hydroxide, can also be employed in the subcutaneous formulation.

Typically, a pH range for the subcutaneous formulation can be in the range of from about 5 to about 12. A preferred pH range for subcutaneous formulation wherein the HDAC inhibitor has a hydroxamic acid moiety, can be about 9 to about 12. Consideration should be given to the solubility and chemical compatibility of the HDAC inhibitor in choosing an appropriate excipient.

The HDAC inhibitors can also be administered in intranasal form via topical use of suitable intranasal vehicles, or via transdermal routes, using those forms of transdermal skin patches well known to those of ordinary skill in that art. To be administered in the form of a transdermal delivery system, the dosage administration will, or course, be continuous rather than intermittent throughout the dosage regime.

In the treatment of rheumatoid arthritis the HDAC inhibitor can be administered directly into the synovial fluid and/or synovial tissue of the rheumatic joint such that a local effect of the inhibitor is realized.

The HDAC inhibitors can be administered as active ingredients in admixture with suitable pharmaceutical diluents, excipients or carriers (collectively referred to herein as “carrier” materials) suitably selected with respect to the intended form of administration, that is, oral tablets, capsules, elixirs, syrups and the like, and consistent with conventional pharmaceutical practices.

For instance, for oral administration in the form of a tablet or capsule, the HDAC inhibitor can be combined with an oral, non-toxic, pharmaceutically acceptable, inert carrier such as lactose, starch, sucrose, glucose, methyl cellulose, microcrystalline cellulose, sodium croscarmellose, magnesium stearate, dicalcium phosphate, calcium sulfate, mannitol, sorbitol and the like or a combination thereof; for oral administration in liquid form, the oral drug components can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn-sweeteners, natural and synthetic gums such as acacia, tragacanth or sodium alginate, carboxymethylcellulose, microcrystalline cellulose, sodium croscarmellose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch methyl cellulose, agar, bentonite, xanthan gum and the like.

Suitable pharmaceutically acceptable salts of the histone deacetylase compounds described herein and suitable for use in the method of the invention, are conventional non-toxic salts and can include a salt with a base or an acid addition salt such as a salt with an inorganic base, for example, an alkali metal salt (e.g. lithium salt, sodium salt, potassium salt, etc.), an alkaline earth metal salt (e.g. calcium salt, magnesium salt, etc.), an ammonium salt; a salt with an organic base, for example, an organic amine salt (e.g. triethylamine salt, pyridine salt, picoline salt, ethanolamine salt, triethanolamine salt, dicyclohexylamine salt, N,N′-dibenzylethylenediamine salt, etc.) etc.; an inorganic acid addition salt (e.g. hydrochloride, hydrobromide, sulfate, phosphate, etc.); an organic carboxylic or sulfonic acid addition salt (e.g. formate, acetate, trifluoroacetate, maleate, tartrate, methanesulfonate, benzenesulfonate, p-toluenesulfonate, etc.); a salt with a basic or acidic amino acid (e.g. arginine, aspartic acid, glutamic acid, etc.) and the like.

When the histone deacetylase inhibitors are used in a method of reducing the level or activity of TRX in a cell comprising contacting the cell with a compound capable of inhibiting a histone deacetylase or a salt thereof in an amount effective to reduce the level of TRX the cell can be a transgenic cell. In another embodiment the cell can be in a subject, such as a mammal, for example a human.

In certain embodiments, the amount effective to reduce the level of thioredoxin in a cell is a contact concentration of HDAC inhibitor from about 1 pM to about 50 μM such as, from about 1 pM to about 5 μM, for example, from about 1 pM to about 500 nM, such as from about 1 pM to about 50 mM, for example, 1 pM to about 500 pM. In a particular embodiment, the concentration is less than about 5.0 μM. In another embodiment, the concentration is about 500 nM.

It is understood that the standard therapy agent, which can be administered in combination with the HDAC inhibitors suitable for use in the invention, can be administered by the methods described above for the HDAC inhibitors. The combination of agents, however, can be administered using the same or different methods.

The following examples are presented in order to more fully illustrate the preferred embodiments of the invention. They should in no way be construed, however, as limiting the broad scope of the invention.

Experimental Methods

Overview

It has been determined, employing microarray analysis, that SAHA induces the expression of the thioredoxin-binding protein-2 (TBP-2) gene in LNCaP prostate cells, and MCF-7 and MDA-MB-468 breast cells. The induction of TBP-2 was associated with a decrease in thioredoxin (TRX) mRNA levels in these cells. It has also been determined that TBP-2 mRNA levels are reduced in human breast and colon tumor tissue compared with matched samples of normal tissues. The promoter region of the TBP-2 gene was cloned and it has been determined that it is directly responsive to SAHA.

Procedures

Cell Culture:

LNCaP prostate carcinoma, T24 bladder carcinoma, ARP-1 myeloma, MCF7 and MDA-MB-468 breast carcinoma cells were obtained from the American Type Culture Collection and cultured as suggested. SAHA was synthesized as described previously (Richon, V. M., et al., PNAS 93(12):5705-5708, 1996) and was dissolved and diluted in dimethyl sulfoxide.

Microarray Analysis:

LNCaP human prostate carcinoma cells (5×10⁶) were cultured in the presence of solvent alone (dimethyl sulfoxide, DMSO) or SAHA (7.5 μM) for 0.5, 2, 6 or 24 hours and total RNA was isolated from the cells using Trizol reagent (Gibco BRL, Rochester, N.Y.). Poly A+ mRNA was isolated from the total RNA using Oligotex columns (Qiagen, Valencia, Calif.). Poly A+ mRNA from cells cultured with SAHA was compared with mRNA from cells cultured without SAHA using the UniGEM human cDNA array (Incyte, St. Louis, Mo.). A 2-fold change was considered as a threshold for determining differences in gene expression.

Northern Blotting:

Total RNA (10 mg) was analyzed by Northern blotting using a ³²P-labeled 1.1 kb TBP-2 coding region cDNA probe, or a 500 bp cDNA probe for human TRX according to Ausubel et al. (Ausubel, F. A. et al., Current Protocols in Molecular Biology, John Wiley, New York, 1998).

Expression of TBP-2 in Normal and Tumor Tissues

Northern blots containing poly A+ mRNA extracted from a panel of different normal human tissues was obtained from Clontech (Clontech, Palo Alto, Calif.). Blots were hybridized first with a ³²P-labelled 1.1-kb TBP-2 cDNA probe, then with a ³²P-labelled 2.0 kb cDNA probe for b-actin, as a loading control. Dot blots of cDNAs from matched pairs of normal and tumor tissues from human patients were obtained from Clontech. The manufacturer (Clontech) normalized the quantities of cDNA on the blot using three housekeeping genes: ubiquitin, 23-kDA highly basic protein and ribosomal protein S9. The blot was hybridized with the ³²P-labelled 1.1-kb TBP-2 cDNA probe according to the manufacturer's instructions.

Cloning of the 5′ Regulatory Region of the TBP-2 Gene

Rapid amplification of cDNA ends (RACE) was performed to determine the transcriptional start site of the TBP-2 gene, using TBP-2 specific primer 1: 5′-GTTGGTTTTAAGAGTTAGAAATGACGG-3 and nested primer 2: 5′-TAAGGTATTCTTAAGCAGTTTGAGC-3 with the Marathon-ready human brain cDNA (Clontech), according to the manufacturer's instructions. A product of approximately 200 bp was amplified, gel-purified, subcloned and nine independent clones were sequenced. From this sequence, two additional primers (5′CCAATTGCTGGAGAAAAGATCCG-3′ and 5′AAGATCCGATCTCCACAAGC ACTCC-3′) were designed. These two primers were used to clone the promoter of the TBP-2 gene by genome walking using the GenomeWalker kit (Clontech). Products ranging from approximately 1200-1800 bp were amplified from three of the genomic libraries (SspI PvuII and DraI), gel-purified and subcloned for sequencing. Sequencing was performed at the DNA Sequencing Facility at Cornell University (Ithaca, N.Y.). At least 2 clones obtained from each of the 3 libraries were sequenced and all were found to be virtually identical in the region directly upstream of the 5′UTR of the TBP-2 gene. The sequence for the 1763 bp SspI fragment was deposited in GenBank (accession number AF408392). During the preparation of this manuscript, the sequence of the TBP-2 gene was deposited into the GenBank database (accession numbers AB051901 and AF333001) and the Human Genome database (accession number NT-004883.4).

Luciferase Assays for Analysis of TBP-2 Promoter Activity Construction of TBP-2/PGL2-Luc Vectors

The 1763 bp SspI fragment was subcloned into the pGL-2 luciferase reporter vector (Promega, Madison, Wis.) to make the pTBP-2-(-2026)-Luciferase construct.

Deletion Constructs of the TBP-2 Promoter

Deletion constructs of the TBP-2 promoter sequence were generated by PCR cloning. The following primers with the original nested TBP-2 specific primer end at −264 bp from the translation initiation site were used to amplify the TBP-2 promoter from the −2026 (1763 bp) construct to generate 5′ deletion mutants:

```
1049: 5′-TGAGCTCAACAGCACAGGCACGCAGCC-3′,
  901: 5′-TGAGCTCCAAGAGAAGGACAAAGGGC-3′
  784: 5′-TGAGCTCGCCAGGAATAACGACAGGC-3′,
  679: 5′-TGAGCTCCAGAAACGTCCACACCCG-3′,
  604: 5′-TGAGCTCCTGGACCCGGGAOAAGACG-3′,
  530: 5′-TGAGCTCTGCGCCGCTCCAGAGCGC-3′,
  482: 5′-TGAGCTCGTGTCCACGCGCCACAGCG-3′,
  440: 5′-TGAGCTCTGGTAAACAAGGACCGGG-3′,
  395: 5′TGAGCTCGCAGCACGAGCCTCCGGG-3′,
  349: 5′TGAGCTCGGCTACTATATAGAGACG-3′.
```

All of the above primers contained NheI sites (underlined) to facilitate cloning, and all clones were sequenced prior to analysis.
Generation of a TBP-2 Promoter Construct Containing Mutations in the Inverted CCAAT Box

Point mutations that abolish the inverted CCAAT box site were introduced by PCR-directed mutagenesis (Ausubel, F. A. et al., Current Protocols in Molecular Biology, John Wiley, New York, 1998) using primers 5′-AACAGCACAGGCACGCAGCC-3′ and 5′-AACAGCACAGGCACGCAGCC-3′. The mutations were confirmed by DNA sequencing.

Reporter Gene Assay

293T cells were plated in 24-well plates in triplicate and were transfected with 100 ng of either pGL-2 vector, pGL-2 SV40 promoter vector (positive control containing the SV40 promoter) or the pGL-2-TBP-2 promoter constructs, using the FuGENE 6 transfection reagent (Roche) according to the manufacturer's instructions. Cells were harvested after 24-48 hrs and luciferase activities were measured using the Dual Luciferase Assay System (Promega), according to the manufacturer's instructions. For experiments in which SAHA or other HDAC inhibitors were used, the transfection medium was replaced with fresh medium containing either solvent control (DMSO), SAHA, m-carboxycinnamic acid bishydroxamate (CBHA, 0.5, 1 or 2 μM) or TSA (100 ng/mL), 12 hours after transfection. After an additional 12-24 hours, the cells were harvested and the lysates were analyzed for luciferase activity as described above.

Results

### EXAMPLE 1

### SAHA Induces Expression of TBP-2 in Transformed Cells

To identify genes wherein expression is modified by SAHA, LNCaP human prostate carcinoma cells were cultured in the presence of either DMSO (vehicle control) or 7.5 μM SAHA for 0.5, 2, 6 or 24 hrs, polyA+ mRNA was isolated and cDNA microarray (Incyte) analysis was performed. TBP-2 was the only gene detected that was induced by more than 2.0-fold after 0.5 hrs in culture. The level of expression of this gene remained increased in LNCaP cells cultured with SAHA for at least 24 hrs. TBP-2 was also induced by more than 2-fold by SAHA (5 μM) in two human breast cancer cell lines, MCF7 (estrogen receptor-positive) and MDA-MB-468 (estrogen receptor negative) following 6 hrs of culture by the microarray technique.

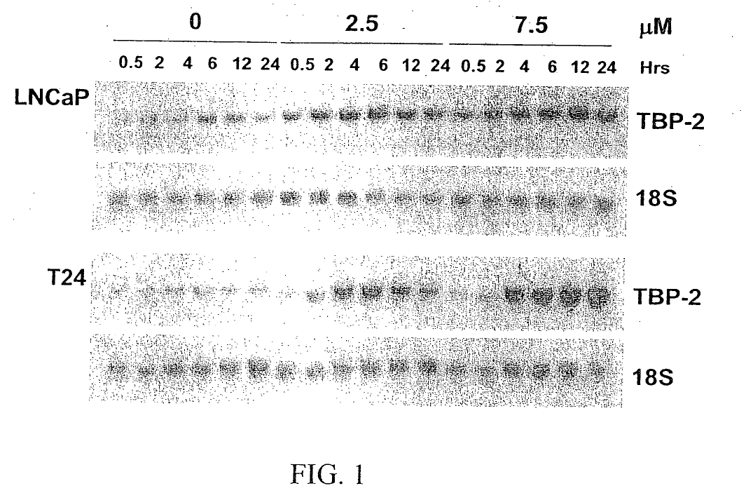

To confirm the microarray results, TBP-2 mRNA levels were analyzed in several transformed cell lines cultured with SAHA by Northern analysis. SAHA (2.5 or 7.5 μM) induced TBP-2 mRNA levels within 2 hours in each transformed cell line examined: T24 bladder carcinoma (FIG. 1), ARP-1 human myeloma, murine erythroleukemia, 293T kidney carcinoma and MCF7 breast carcinoma call lines (data not shown).

### EXAMPLE 2

### Expression of TBP-2 in Normal and Tumor Tissues

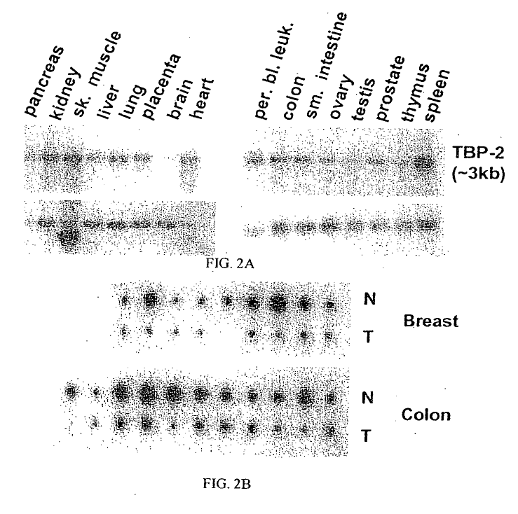

The pattern of expression of TBP-2 mRNA in a panel of 16 normal human tissues was then examined. The highest levels of expression were found in skeletal muscle, kidney and spleen, and the lowest levels of expression in the brain (FIG. 2A).

It was then investigated whether genes whose expression is induced in transformed cells by SAHA are down-regulated during tumorigenesis by analyzing the expression of TBP-2 in normal and tumor tissues. Hybridization of the TBP-2 mRNA expression in colon and breast carcinomas (FIG. 2B).

### EXAMPLE 3

### SAHA Reduces TRX mRNA Levels in Transformed Cells

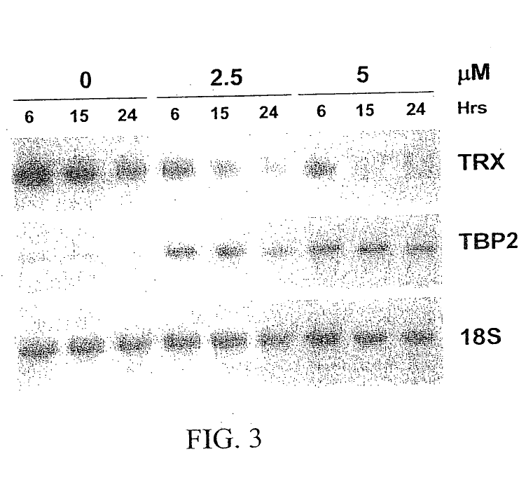

TBP-2 has been identified as a protein that associates with the active (reduced) form of TRX, a dithiol-reducing redox regulatory protein (Nishiyama, A. et al., J. Biol. Chem. 274(31):21645-50, 1999). Binding of TBP-2 to TRX inhibits both the thiol reducing activity and the level of expression of TRX. To determine whether induction of TBP-2 by SAHA is associated with reduced TRX levels, Northern blot analysis of RNA prepared from T24 cultured with SAHA (2.5 and 5 μM), using a full-length TRX cDNA probe was performed. A decrease in the levels of TRX mRNA was observed within 15 hours of culture with SAHA accompanied by a concomitant increase in the level of TBP-2 mRNA (FIG. 3). A similar decrease in TRX mRNA and increase in TBP-2mRNA was detected in ARP-1 and MCF7 cells following culture with SAHA (data not shown).

### EXAMPLE 4

### SAHA Induces TBP-2 Promoter Activity

Cloning and Characterization of the TBP-2 Promoter

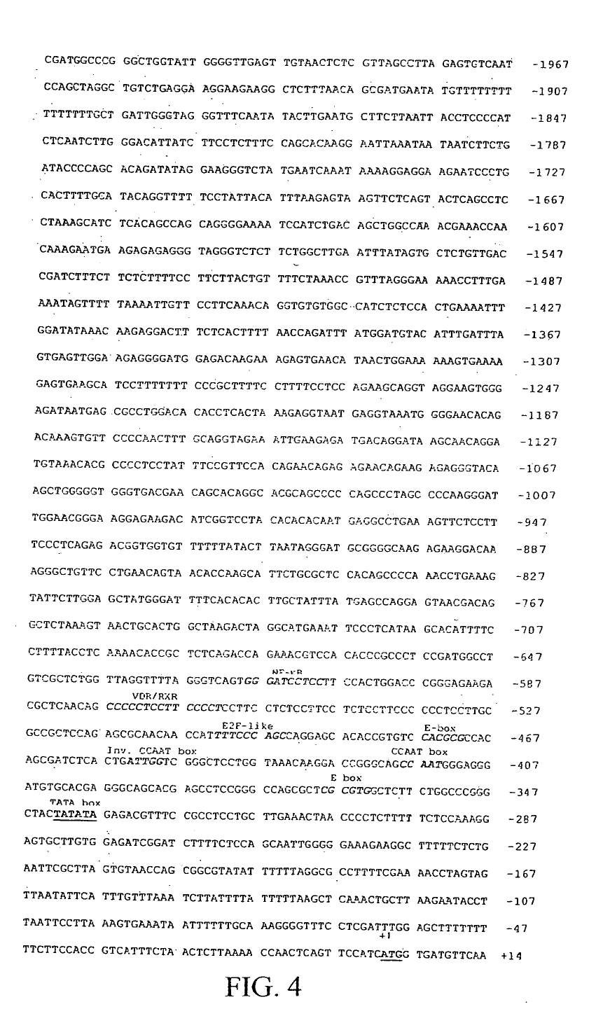

To investigate the mechanism by which SAHA induces the expression of TBP-2 mRNA, the TBP-2 promoter region was cloned using a combination of 5′-RACE and Genome Walking (FIG. 4). The promoter sequence was analyzed using the MatInspector Professional program (http://genomatix.gsf.de) for the presence of classical promoter features. The presence of a putative TATA box as well as putative binding sites for the transcription factors, such as, NF-Y, Myc-Max, E2F, vitamin D receptor/retinoid X receptor and NF-6B were identified (FIG. 4). The Proscan computer program (Version 1.7, http://bimas.dcrt.nih.gov/molbio/proscan/) predicted that a TATA box existed at the same location predicted by MatInspector.

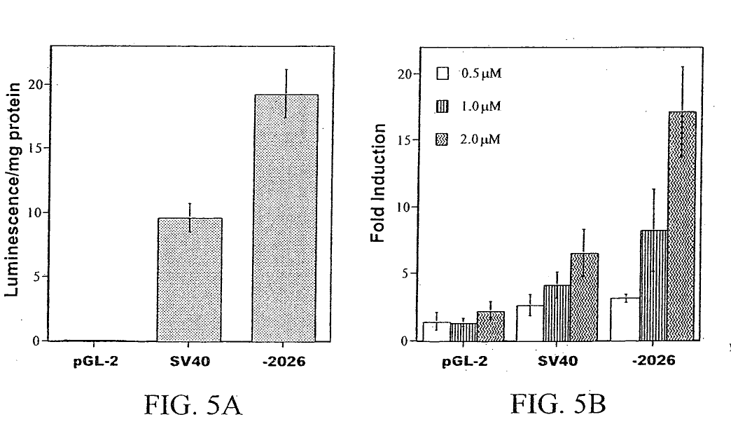

To confirm that the sequence identified by genome walking has promoter activity, the obtained sequence was cloned into a promoter-less pGL-2 luciferase reporter vector and luciferase reporter assays were performed. Transfection of the pGL-2-TBP-2 construct, −2026, into 293T cells yielded reporter gene activity equivalent to or greater than the SV40 positive control promoter while transfection with pGL-2 vector yielded barely detectable reporter gene activity (FIG. 5A), indicating that the cloned TBP-2 promoter is functional.

Effect of SAHA on Cloned TBP-2 Promoter:

To determine SAHA ability to induce the activity of the cloned TBP-2 promoter, 293T cells were transfected with reporter constructs and cultured with SAHA. The activity of the TBP-2 promoter fragment was induced in a dose-dependent manner by SAHA (FIG. 5B). The activity of the SV40 control promoter was induced by SAHA, but not to the same extent as the TBP-2 promoter (FIG. 5B). The activity of the TBP-2 promoter was also induced by m-carboxycinnamic acid bishydroxamic acid (CBHA) a related hydroxamic acid-based hybrid polar inhibitor of HDAC activity (data not shown).

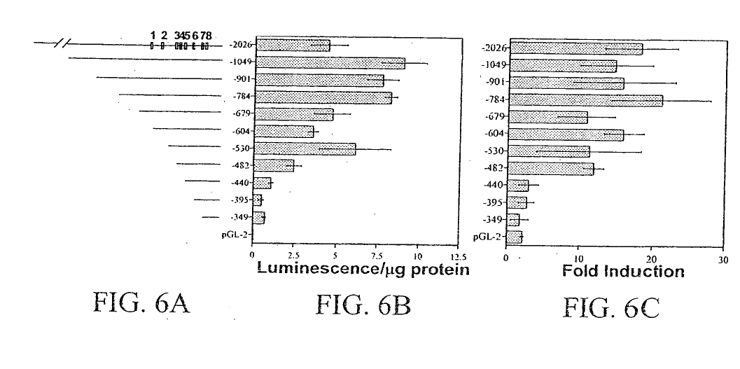

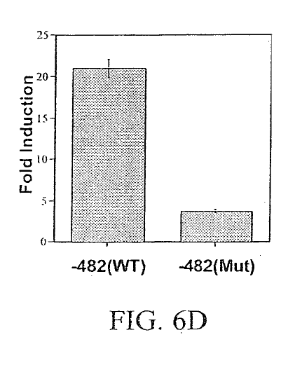

To determine which potential transcription factor binding sites are important for TBP-2 gene transcription and induction by SAHA, a series of deletion constructs were generated (FIG. 6A). Transient transfection assays showed that promoter constructs −2026 to −482 had comparable luciferase activity (FIG. 6B). With further deletion of the promoter region, there was a loss of promoter activity (FIG. 6B). Addition of SAHA (2 μM) to transfected cells caused an induction of luciferase activity of 12 to 20-fold after 24 hrs of cultures, for promoter constructs −2026 to −482 (FIG. 6C). However, promoter constructs −440 to −349 showed reduced levels of induction (2 to 3-fold) in response to SAHA (FIG. 6C). This suggested the presence of a site between promoter constructs −482 and −440 that is critical for optimal induction of TBP-2 by HDAC inhibitors. This region of the promoter contains putative E-box and inverted CCAAT box sites. Several transcription factors, including NF-Y (Mantovani, R., Nucleic Acids Res. 26(5):1135-43, 1998) bind to the inverted CCAAT box. Induction of the multidrug resistance 1 gene (MDR1) (Jin, S. et al., Mol. Cell Biol. 18(7):4377-84, 1998) and the SHP-1 gene (Xu, Y. et al., Gene 269(1-2):141-53, 2001) promoters by the HDAC inhibitors TSA and/or butyrate requires the presence of a functional NF-Y binding site. We introduced two point mutations into the inverted CCAAT box (ATTGG-ATGTG) and generated pGL-2 luciferase constructs to test the activity and SAHA inducibility of the inverted CCAAT box mutant promoter. The −482 construct consisting of the mutated inverted CCAAT box showed a lower level of induction (3.7-fold) by SAHA than the wild-type −482 construct which was induced 21-fold (FIG. 6D). These results indicate that the inverted CCAAT box in the TBP-2 promoter is critical for the optimal induction of the TBP-2 promoter by SAHA.

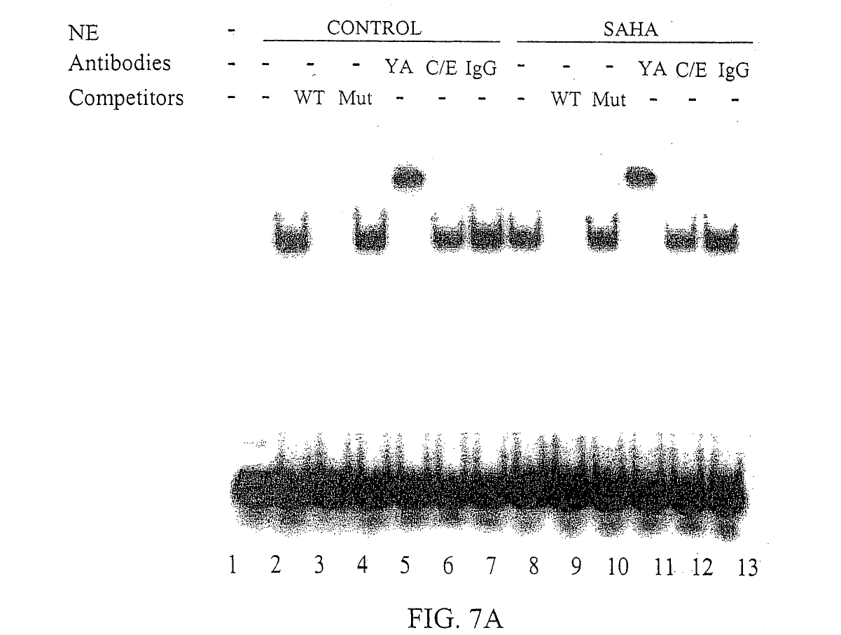

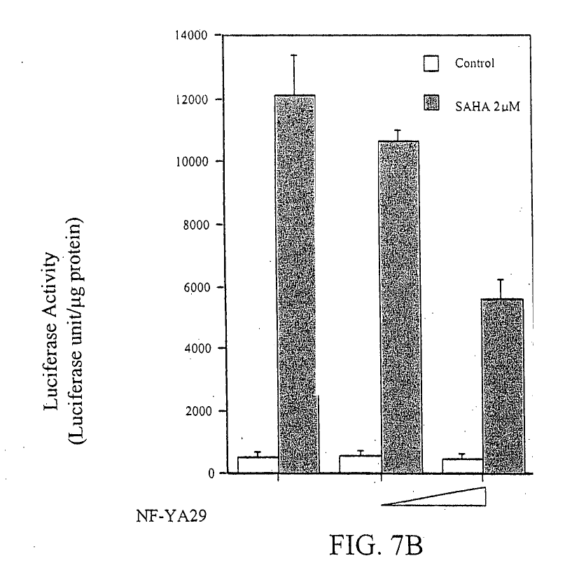

Electrophoretic mobility-shift assays using nuclear extracts prepared from control and SAHA-treated T24 cells were performed to determine whether NF-Y binds this inverted CCAAT box in the TBP-2 promoter. A specific protein-DNA complex was detected (FIG. 7A, lanes 2 and 8). The wild-type unlabeled competitor blocked the formation of the complex (FIG. 7A, lanes 3 and 9), but the inverted CCAAT box mutant competitor had no effect (FIG. 7A, lanes 4 and 10). Supershift analysis was then performed to identify the proteins bound to the inverted CCAAT box. The polyclonal antibody against NF-YA resulted in a supershift of the protein-DNA complex, whereas an antibody against another CCAAT box binding protein C/EBP did not alter the mobility of the complex. These results indicate that the inverted CCAAT box site in the TBP-2 promoter is capable of binding NF-Y. Similar results were observed from the nuclear extracts of untreated (FIG. 7A, lanes 2-7) and cells cultured with SAHA (FIG. 7A, lanes 8-13).

To further investigate the role of NF-Y in SAHA induction of the TBP-2 promoter, a dominant negative NF-YA mutant expression vector, NF-YA29, was cotransfected with −2026 pGL2-TBP-2 promoter construct into 293T cells. NF-Y29 is a dominant negative form of NF-YA with a mutation of 3 amino acids in the DNA binding domain. NF-Y29 forms a complex with NF-YB (and NF-YC), but fails to bind the CCAAT box (29). NF-YA29 decreased the TBP-2 promoter induction by SAHA (FIG. 7B). Taken together, these results support a role for NF-Y in the induction of TBP-2 transcription by SAHA.

All references cited herein are incorporated by reference in their entirety. While this invention has been particularly shown and described with references to preferred embodiments thereof, it will be understood by those skilled in the art that various changes in form and details may be made therein without departing from the scope of the invention encompassed by the appended claims.

1.-90. (canceled)

91. A pharmaceutical composition comprising suberoylanilide hydroxamic acid (SAHA), represented by the structure: 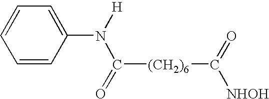
formulated as a pharmaceutical salt with an organic or inorganic base.

92. The pharmaceutical composition of claim 91, wherein the organic base is a base comprising triethylamine, pyridine, picoline, ethanolamine, triethanolamine, dicyclohexylamine, N,N′-dibenzylethylenediamine, or arginine.

93. The pharmaceutical composition of claim 91, wherein the organic base is a base comprising ethanolamine or arginine.

94. The pharmaceutical composition of claim 91, wherein the inorganic base is a base comprising ammonium, alkali metal, or alkaline earth metal.

95. The pharmaceutical composition of claim 91, wherein the inorganic base is a base comprising sodium, potassium, lithium, calcium, or magnesium.

96. The pharmaceutical composition of claim 91, wherein the inorganic base is a base comprising sodium, potassium, or ammonium.

97. The pharmaceutical composition of claim 91, which is formulated as a hydrate.

98. The pharmaceutical composition of claim 91, which is formulated as a hydrate selected from the group consisting of a hemihydrate, monohydrate, dihydrate, trihydrate, and tetrahydrate.

99. The pharmaceutical composition of claim 91, wherein the composition comprises a dose of about 200 mg to about 2000 mg of SAHA formulated as a pharmaceutical salt.

100. The pharmaceutical composition of claim 91, wherein the composition comprises a dose of about 200 mg to about 1200 mg of SAHA formulated as a pharmaceutical salt.

101. The pharmaceutical composition of claim 91, wherein the composition comprises a dose of about 400 mg to about 1200 mg of SAHA formulated as a pharmaceutical salt.

102. The pharmaceutical composition of claim 91, wherein the composition comprises a dose of about 200 mg of SAHA formulated as a pharmaceutical salt.

103. The pharmaceutical composition of claim 91, wherein the composition comprises a dose of about 400 mg of SAHA formulated as a pharmaceutical salt.

104. The pharmaceutical composition of claim 91, which is formulated for oral administration.

105. The pharmaceutical composition of claim 92, which is formulated for oral administration.

106. The pharmaceutical composition of claim 93, which is formulated for oral administration.

107. The pharmaceutical composition of claim 94, which is formulated for oral administration.

108. The pharmaceutical composition of claim 95, which is formulated for oral administration.

109. The pharmaceutical composition of claim 96, which is formulated for oral administration.

110. The pharmaceutical composition of claim 97, which is formulated for oral administration.

111. The pharmaceutical composition of claim 98, which is formulated for oral administration.

112. The pharmaceutical composition of claim 99, which is formulated for oral administration.

113. The pharmaceutical composition of claim 100, which is formulated for oral administration.

114. The pharmaceutical composition of claim 101, which is formulated for oral administration.

115. The pharmaceutical composition of claim 102, which is formulated for oral administration.

116. The pharmaceutical composition of claim 103, which is formulated for oral administration.


SEQUENCE LISTING (18 sequences)

SEQ ID NO: 1 - protein, 4 aa, homo sapiens
CGPC

SEQ ID NO: 2 - DNA, 2040 bp, homo sapiens
cgatggcccgggctggtattggggttgagttgtaactctcgttagccttagagtgtcaatccagctaggctgtctgaggaaggaagaaggctctttaacagcgatgaatatgtttttttttttttttgctgattgggtagggtttcaatatacttgaatgcttcttaattacctccccatctcaatcttgggacattatcttcctctttccagcacaaggaattaaataataatcttctgataccccagcacagatataggaagggtctatgaatcaaataaaaggaggaagaatccctgcacttttgcatacaggtttttcctattacatttaagagtaagttctcagtactcagcctcctaaagcatctcacagccagcaggggaaaatccatctgacagctggccaaacgaaaccaacaaagaatgaagagagagggtagggtctcttctggcttgaatttatagtgctctgttgaccgatctttcttctcttttccttcttactgttttctaaaccgtttagggaaaaacctttgaaaatagtttttaaaattgttccttcaaacaggtgtgtggccatctctccactgaaaatttggatataaacaagaggactttctcacttttaaccagatttatggatgtacatttgatttagtgagttggaagaggggatggagacaagaaagagtgaacataactggaaaaaagtgaaaagagtgaagcatcctttttttcccgcttttccttttcctccagaagcaggtaggaagtgggagataatgagcgcctggacacacctcactaaagaggtaatgaggtaaatggggaacacagacaaagtgttccccaactttgcaggtagaaattgaagagatgacaggataagcaacaggatgtaaacacgcccctcctatttccgttccacagaacagagagaacagaagagagggtacaagctgggggtgggtgacgaacagcacaggcacgcagcccccagccctagccccaagggattggaacgggaaggagaagacatcggtcctacacacacaatgaggcctgaaagttctcctttccctcagagacggtggtgttttttatgcttaatagggatgcggggcaagagaaggacaaagggctgttcctgaacagtaacaccaagcattctgcgctccacagccccaaacctgaaagtattcttggagctatgggattttcacacacttgctatttatgagccaggagtaacgacaggctctaaagtaactgcactggctaagactaggcatgaaattccctcataagcacattttccttttacctcaaaacaccgctctcagaccagaaacgtccacacccgccctccgatggcctgtcgctctggttaggttttagggtcagtgggatcctccttccactggacccgggagaagacgctcaacagccccctccttcccctccttcctctccttcctctccttcccccctccttgcgccgctccagagcgcaacaaccattttcccagccaggagcacaccgtgtccacgcgccacagcgatctcactgattggtcgggctcctggtaaacaaggaccgggcagccaatgggagggatgtgcacgagggcagcacgagcctccgggccagcgctcgcgtggctcttctggcccgggctactatatagagacgtttccgcctcctgcttgaaactaacccctctttttctccaaaggagtgcttgtggagatcggatcttttctccagcaattgggggaaagaaggctttttctctgaattcgcttagtgtaaccagcggcgtatattttttaggcgccttttcgaaaacctagtagttaatattcatttgtttaaatcttattttatttttaagctcaaactgcttaagaataccttaattccttaaagtgaaataattttttgcaaaggggtttcctcgatttggagctttttttttcttccaccgtcatttctaactcttaaaaccaactcagttccatcatggtgatgttcaa

SEQ ID NO: 3 - DNA, 27 bp, homo sapiens
gttggttttaagagttagaaatgacgg

SEQ ID NO: 4 - DNA, 25 bp, homo sapiens
taaggtattcttaagcagtttgagc

SEQ ID NO: 5 - DNA, 23 bp, homo sapiens
ccaattgctggagaaaagatccg

SEQ ID NO: 6 - DNA, 25 bp, homo sapiens
aagatccgatctccacaagcactcc

SEQ ID NO: 7 - DNA, 27 bp, homo sapiens
tgagctcaacagcacaggcacgcagcc

SEQ ID NO: 8 - DNA, 26 bp, homo sapiens
tgagctccaagagaaggacaaagggc

SEQ ID NO: 9 - DNA, 26 bp, homo sapiens
tgagctcgccaggaataacgacaggc

SEQ ID NO: 10 - DNA, 25 bp, homo sapiens
tgagctccagaaacgtccacacccg

SEQ ID NO: 11 - DNA, 26 bp, homo sapiens
tgagctcctggacccgggagaagacg

SEQ ID NO: 12 - DNA, 25 bp, homo sapiens
tgagctctgcgccgctccagagcgc

SEQ ID NO: 13 - DNA, 26 bp, homo sapiens
tgagctcgtgtccacgcgccacagcg

SEQ ID NO: 14 - DNA, 25 bp, homo sapiens
tgagctctggtaaacaaggaccggg

SEQ ID NO: 15 - DNA, 25 bp, homo sapiens
tgagctcgcagcacgagcctccggg

SEQ ID NO: 16 - DNA, 25 bp, homo sapiens
tgagctcggctactatatagagacg

SEQ ID NO: 17 - DNA, 20 bp, homo sapiens
aacagcacaggcacgcagcc

SEQ ID NO: 18 - DNA, 20 bp, homo sapiens
aacagcacaggcacgcagcc